(19) United States
(12) Patent Application Publication
(10) Pub. No.: US 2018/0296988 A1
(43) Pub. Date: Oct. 18, 2018

(54) CELL-TRAPPING FILTER

(71) Applicants: Asahi Glass Company, Limited, Chiyoda-ku (JP); National University Corporation Nagoya University, Nagoya-shi (JP)

(72) Inventors: Naoto KIHARA, Chiyoda-ku (JP); Ryohei KOGUCHI, Chiyoda-ku (JP); Hajime EGUCHI, Chiyoda-ku (JP); Masaru HORI, Nagoya-shi (JP); Hiromasa TANAKA, Nagoya-shi (JP); Kenji ISHIKAWA, Nagoya-shi (JP); Yoshinobu BABA, Nagoya-shi (JP); Hiroshi YUKAWA, Nagoya-shi (JP); Daisuke ONOSHIMA, Nagoya-shi (JP); Tetsunari HASE, Nagoya-shi (JP); Daiki KUBOYAMA, Nagoya-shi (JP)

(73) Assignees: Asahi Glass Company, Limited, Chiyoda-ku (JP); National University Corporation Nagoya University, Nagoya-shi (JP)

(21) Appl. No.: 16/014,463

(22) Filed: Jun. 21, 2018

Related U.S. Application Data
(63) Continuation of application No. PCT/JP2016/078095, filed on Sep. 23, 2016.

(30) Foreign Application Priority Data
Dec. 24, 2015 (JP) 2015-252237

(51) Int. Cl. B01D 71/34; B01D 71/36; B01D 71/50; B01D 71/04; B01D 69/02; C12M 1/00

(52) U.S. Cl. CPC B01D 71/34; B01D 71/36; B01D 71/50; B01D 2325/38; B01D 69/02; C12M 47/04; B01D 71/04

(57) ABSTRACT

To provide a cell-trapping filter which has a high cell trapping efficiency and which is excellent in water resistance.

A cell-trapping filter comprising a substrate and a cell-separating mechanism by size, wherein the substrate has, at least on its surface, a layer formed of a fluorinated polymer having units having a biocompatible group, having a fluorine atom content of from 5 to 60 mass % and having a proportion P represented by the following formula of from 0.1 to 5%:

proportion P=(proportion (mass %) of units having biocompatible group to all units in fluorinated polymer/fluorine atom content (mass %) of fluorinated polymer)×100

### TECHNICAL FIELD

The present invention relates to a cell-trapping filter, and a method for trapping cells by using it.

### BACKGROUND ART

Heretofore, a method of using a substrate having a number of holes disposed thereon, to trap single cells in the holes and separate them, and a method of analyzing the trapped single cells have been known (Patent Documents 1 and 2).

Further, a method of covering the surface of the substrate or the inner wall of the holes with a hydrophilic polymer material to conduct hydrophilic treatment (Patent Document 3) and a method of covering them with a polymer material having nonionic surface activity (Patent Document 4) have been known. By these methods, an improvement of the cell-trapping efficiency can be expected.

However, in the above methods, since the polymer material is water-soluble, if a coating layer is formed on the substrate using the polymer material alone, the polymer material may be eluted from the coating layer during use to be cytotoxin or to be impurities in the subsequent analysis, thus leading to problems.

### PRIOR ART DOCUMENTS

### Patent Documents

Patent Document 1: JP-A-2012-177686

Patent Document 2: Japanese Patent No. 5081854

Patent Document 3: Japanese Patent No. 5487152

Patent Document 4: Japanese Patent No. 5704590

### DISCLOSURE OF INVENTION

### Technical Problem

The object of the present invention is to provide a cell-trapping filter which has a high cell-trapping efficiency and which is excellent in the water resistance, and a method for trapping cells by using the cell-trapping filter.

### Solution to Problem

The present invention provides a cell-trapping filter comprising a substrate and a cell-separating mechanism by size, wherein the substrate has, on its surface, a layer formed of a fluorinated polymer having units having a biocompatible group, having a fluorine atom content of from 5 to 60 mass % and having a proportion P represented by the following formula of from 0.1 to 5%:

proportion P=(proportion (mass %) of units having biocompatible group to all units in fluorinated polymer/fluorine atom content (mass %) of fluorinated polymer)×100

### Advantageous Effects of Invention

The cell-trapping filter of the present invention has a high cell-trapping efficiency due to low adsorption of protein, and is excellent in water resistance. According to the cell-trapping filter, it is possible to prevent problems caused by cytotoxin or impurities in the subsequent analysis derived from a polymer material eluted during use of the cell-trapping filter.

### DESCRIPTION OF EMBODIMENTS

The following definitions of terms apply throughout the present specification including claims.

A “fluorinated polymer” means a polymer compound having fluorine atom(s) in the molecule.

A “glass transition temperature (Tg)” of a polymer means a temperature for a change from the rubbery state to the glass state, as measured by a differential scanning calorimetry (DSC) method.

A “unit” means a moiety derived from a monomer, which is present in a polymer and which constitutes the polymer. A unit derived from a monomer having a carbon-carbon unsaturated double bond, formed by addition polymerization of the monomer, is a divalent unit formed by cleavage of the unsaturated double bond. Further, one obtained by chemically converting the structure of a certain unit after formation of a polymer will also be referred to as a unit. In the following, in some cases, a unit derived from an individual monomer may be referred to by a name having “unit” attached to the monomer's name.

A “(meth)acrylate” is a generic term for an acrylate and a methacrylate.

A “biocompatible group” means a group having a property of inhibiting adsorption of protein on a polymer and adhesion and fixing of cells on a polymer.

A “segment” means a molecular chain formed by two or more units which are chained.

The term “biocompatibility” means a property not to let protein be adsorbed, or not to let cells be adhered.

A “cell” is the most fundamental unit constituting a living body and means one which has, in the interior of the cell membrane, the cytoplasm and various organelles. Nuclei containing DNA may be contained or may not be contained inside the cell.

Animal-derived cells include germ cells (sperm, ova, etc.), somatic cells constituting a living body, stem cells, progenitor cells, cancer cells separated from a living body, cells (cell line) which are separated from a living body and have won immortalized ability and thus are stably maintained outside the body, cells separated from a living body and artificially genetically engineered, cells separated from a living body and having nuclei artificially replaced, etc.

Somatic cells constituting a living body include fibroblasts, bone marrow cells, B lymphocytes, T lymphocytes, neutrophils, erythrocytes, platelets, macrophages, monocytes, bone cells, bone marrow cells, pericytes, dendritic cells, keratinocytes, fat cells, mesenchymal cells, epithelial cells, epidermal cells, endothelial cells, vascular endothelial cells, hepatocytes, cartilage cells, cumulus cells, neural cells, glial cells, neurons, oligodendrocytes, microglia, astrocytes, cardiac cells, esophagus cells, muscle cells (for example, smooth muscle cells, skeletal muscle cells), pancreatic beta cells, melanin cells, hematopoietic progenitor cells, mononuclear cells, etc.

The stem cells are cells having both an ability to replicate themselves and an ability to be differentiated into cells of other multiple systems, and include embryonic stem cells (ES cells), embryonic carcinoma cells, embryonic germ stem cells, induced pluripotent stem cells (iPS cells), neural stem cells, hematopoietic stem cells, mesenchymal stem cells, liver stem cells, pancreatic stem cells, muscle stem cells, germ stem cells, intestinal stem cells, cancer stem cells, hair follicle stem cells, etc.

The progenitor cells are cells at an intermediate stage during differentiation into specific somatic or germ cells from the stem cells.

The cancer cells are cells that have acquired an unlimited proliferative capacity as derived from somatic cells.

A cell line is cells which have acquired an unlimited proliferative capacity by an artificial manipulation in vitro, and includes HCT116, Huh7, HEK293 (human embryonic kidney cells), HeLa (human cervical carcinoma cell line), HepG2 (human liver cancer cell line), UT7/TPO (human leukemia cell line), CHO (Chinese hamster ovary cell line), MDCK, MDBK, BHK, C-33A, HT-29, AE-1, 3D9, Ns0/1, Jurkat, NIH3T3, PC12, S2, Sf9, Sf21, High Five, Vero, etc.

CTCs (Circulating Turnor Cells) are cancer cells present in the blood of a cancer patient. CAMLs (Circulating Cancer Associated Macrophage-like Cells) are macrophage-like cells present in the blood of a cancer patient.

In this specification, a group represented by the formula (1) will be referred to as a group (1). Groups represented by other formulae will be referred to in the same manner.

### (Fluorinated Polymer)

The fluorinated polymer in the present invention (hereinafter referred to also as “fluorinated polymer (A)”) is a fluorinated polymer which has units having a biocompatible group, a fluorine atom content of from 5 to 60 mass % and a proportion P represented by the following formula of from 0.1 to 5%. According to the cell-trapping filter of the present invention, which has a layer formed of the fluorinated polymer (A) on the substrate surface, deposition of proteins can be prevented. And, the layer formed of the fluorinated polymer (A) is excellent in the water resistance.

Proportion P=(proportion(mass %) of units having biocompatible group to all units in fluorinated polymer(A)/fluorine atom content(mass %) in fluorinated polymer(A))×100

### <Biocompatible Group>

The biocompatible group is preferably at least one member selected from the group consisting of the following group (1), group (2) and group (3), from such a viewpoint that it is thereby easy to form a coating layer whereby the effect to prevent adsorption of protein is high. As the biocompatible group, from such a viewpoint that it is thereby easy to obtain the effect to prevent adsorption of protein, preferred is the group (1) only, or one or both of the group (2) and the group (3), and particularly preferred is either one of the group (1), the group (2) or the group (3). The fluorinated polymer (A) is excellent in biocompatibility when it contains at least one member selected from the group consisting of the group (1), the group (2) and the group (3).

CₙH₂ₙOₘ (1)

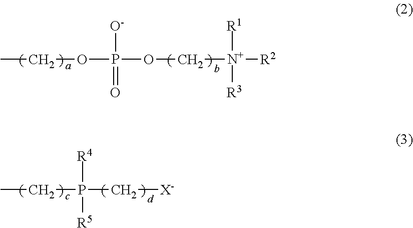

Here, in the above formulae, n is an integer of from 1 to 10, m is an integer of from 1 to 100 in a case where the group (1) is contained in a side chain in the fluorinated polymer (A) or from 5 to 300 in a case where contained in the main chain, R¹ to R³ are each independently a C₁₋₅ alkyl group, “a” is an integer of from 1 to 5, b is an integer of from 1 to 5, R⁴ and R⁵ are each independently a C₁₋₅ alkyl group, X⁻ is the following group (3-1) or the following group (3-2), c is an integer of from 1 to 20, d is an integer of from 1 to 5.

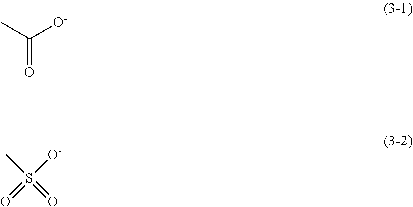

### Group (1):

The group (1) has a high mobility in blood, etc., whereby protein is less likely to be adsorbed on the surface of the coating layer.

The group (1) may be contained in the main chain of the fluorinated polymer (A), or it may be contained in a side chain.

n in the group (1) is preferably an integer of from 1 to 6, more preferably an integer of from 1 to 4, from such a viewpoint that protein is thereby less likely to be adsorbed. The group (1) may be linear or branched. From the viewpoint of a higher effect to prevent adsorption of protein, the group (1) is preferably linear.

When the group (1) is contained in a side chain of the fluorinated polymer (A), m in the group (1) is preferably from 1 to 40, particularly preferably from 1 to 20, from the viewpoint of excellent water resistance. When the group (1) is contained in the main chain of the fluorinated polymer (A), m is preferably from 5 to 300, particularly preferably from 10 to 200, from the viewpoint of excellent water resistance.

When m is 2 or more, the plurality of (CₙH₂ₙO) in the group (1) may be of one type, or may be of two or more types. Further, in the case of two or more types, their disposition may be any of random, block and alternating. When n is 3 or more, (CₙH₂ₙO) may be a straight-chain structure or a branched structure.

In a case where the fluorinated polymer (A) has groups (1), the groups (1) in the fluorinated polymer (A) may be of one type, or of two or more types.

### Group (2):

The group (2) has a strong affinity for phospholipids in blood, while its interaction force against plasma proteins is weak. Therefore, by using a fluorinated polymer (A) having groups (2), for example, it is considered that in blood, phospholipids are adsorbed preferentially on the coating layer, and such phospholipids will be self-assembled to form an adsorption layer. As a result, since the surface becomes a structure similar to the vascular endothelial surface, adsorption of proteins, such as fibrinogen, etc., will be suppressed.

The group (2) is preferably contained in a side chain of the fluorinated polymer (A).

R¹ to R³ in the group (2) are each independently a C₁₋₅ alkyl group, and from the viewpoint of easy availability of raw material, preferably a C₁₋₄ alkyl group, particularly preferably a methyl group. “a” in the group (2) is an integer of from 1 to 5, and from the viewpoint of easy availability of raw material, preferably an integer of 2 to 5, particularly preferably 2. b in the group (2) is an integer of 1 to 5, and from such a viewpoint that protein is less likely to be adsorbed, preferably an integer of 1 to 4, particularly preferably 2.

In a case where the fluorinated polymer (A) has groups (2), the groups (2) in the fluorinated polymer (A) may be of one type, or of two or more types.

### Group (3):

By using a fluorinated polymer (A) having groups (3), adsorption of proteins is inhibited for the same reason as in the case of using the fluorinated polymer (A) having groups (2). The group (3) is preferably contained in a side chain of the fluorinated polymer (A).

R⁴ and R⁵ in the group (3) are each independently a C₁₋₅ alkyl group, and from such a viewpoint that protein is less likely to be adsorbed, preferably a C₁₋₄ alkyl group, particularly preferably a methyl group. c in the group (3) is an integer of from 1 to 20, and from such a viewpoint that the fluorinated polymer (A) will be excellent in flexibility, preferably an integer of from 1 to 15, more preferably an integer of from 1 to 10, particularly preferably 2. d in the group (3) is an integer of from 1 to 5, and from such a viewpoint that protein is less likely to be adsorbed, preferably an integer of from 1 to 4, particularly preferably 1.

In a case where the fluorinated polymer (A) has groups (3), the groups (3) in the fluorinated polymer (A) may be of one type, or of two or more types.

Further, in a case where the fluorinated polymer (A) has groups (3), from such a viewpoint that protein is less likely to be adsorbed, it is preferred that the fluorinated polymer (A) has either groups (3) wherein X⁻ is a group (3-1), or groups (3) wherein X⁻ is a group (3-2).

### <Physical Properties of Fluorinated Polymer (A)>

The proportion P of the fluorinated polymer (A) is from 0.1 to 5.0%. When the proportion P is at least the above lower limit value, it is possible to form a coating layer excellent in biocompatibility, on which protein is less likely to be adsorbed. When the proportion P is at most the above upper limit value, it is possible to form a coating layer excellent in water resistance, whereby the fluorinated polymer (A) is less likely to elute in blood, etc.

The proportion P is preferably from 0.1 to 4.7%, particularly preferably from 0.1 to 4.5%.

Here, the proportion P can be measured by the method described in Examples. Further, it can also be calculated from the charged amounts of the monomers and initiator used in the production of the fluorinated polymer (A).

The fluorine atom content of the fluorinated polymer (A) is from 5 to 60 mass %. The fluorine atom content is preferably from 5 to 55 mass %, particularly preferably from 5 to 50 mass %. When the fluorine atom content is at least the above lower limit value, water resistance will be excellent. When the fluorine atom content is at most the above upper limit value, protein will be less likely to be adsorbed.

Here, the fluorine atom content (mass %) is determined by the following formula.

(Fluorine atom content)=[19×NF/MA]×100

In the above formula, NF is the sum of values obtained by multiplying, for every type of units that constitute the fluorinated polymer, the number of fluorine atoms in the unit by the molar ratio of the unit to all units. MA is the sum of values obtained by multiplying, for every type of units that constitute the fluorinated polymer, the total atomic weight of all atoms constituting the unit by the molar ratio of the unit to all units.

As an example, the fluorine atom content of a fluorinated polymer having 50 mol % of tetrafluoroethylene (TFE) units and 50 mol % of ethylene (E) units, will be described as follows.

In the case of such a fluorinated polymer, the value obtained by multiplying the number of fluorine atoms (4) in a TFE unit by the molar ratio (0.5) of the TFE unit to all units, is 2, and the value obtained by multiplying the number of fluorine atoms (0) in an E unit by the molar ratio (0.5) of the E unit to all units, is 0, and therefore, NF becomes to be 2. Further, the value obtained by multiplying the total atomic weight (100) of all atoms constituting the TFE unit, by the molar ratio (0.5) of the TFE unit to all units, is 50, and the value obtained by multiplying the total atomic weight (28) of all atoms constituting the E unit, by the molar ratio (0.5) of the E unit to all units, is 14, and therefore, MA becomes to be 64. Accordingly, the fluorine atom content of the fluorinated polymer becomes to be 59.4 mass %.

Further, the fluorine atom content can be measured by the method described in Examples. It can also be calculated from the charged amounts of the monomers and initiator used in the production of the fluorinated polymer (A).

The number average molecular weight (Mn) of the fluorinated polymer (A) is preferably from 2,000 to 1,000,000, particularly preferably from 2,000 to 800,000. When the number average molecular weight of the fluorinated polymer (A) is at least the above lower limit value, durability will be excellent, and when it is at most the above upper limit value, processability will be excellent.

The mass average molecular weight (Mw) of the fluorinated polymer (A) is preferably from 2,000 to 2,000,000, particularly preferably from 2,000 to 1,000,000. When the mass average molecular weight of the fluorinated polymer (A) is at least the above lower limit value, durability will be excellent, and when it is at most the above upper limit value, processability will be excellent.

The molecular weight distribution (Mw/Mn) of the fluorinated polymer (A) is preferably from 1 to 10, particularly preferably from 1.1 to 5. When the molecular weight distribution of the fluorinated polymer (A) is within the above range, water resistance will be excellent, and protein will be less likely to be adsorbed.

As the fluorinated polymer (A), a commercially available product may be used. Commercially available products may, for example, be the following.

Manufactured by 3M, Novec series:

FC-4430 (nonionic, containing perfluorobutanesulfonic acid groups, surface tension: 21 mN/m),

FC-4432 (nonionic, containing perfluorobutanesulfonic acid groups, surface tension: 21 mN/m), etc.

Manufactured by AGC Seimi Chemical Co., Ltd., Surflon series:

S-241 (nonionic, containing C₁₋₆ perfluoroalkyl groups, surface tension: 16.2 mN/m),

S-242 (nonionic, C₁₋₆ perfluoroalkyl group-containing ethylene oxide adduct, surface tension: 22.9 mN/m),

S-243 (nonionic, C₁₋₆ perfluoroalkyl group-containing ethylene oxide adduct, surface tension: 23.2 mN/m),

S-420 (nonionic, C₁₋₆ perfluoroalkyl group-containing ethylene oxide adduct, surface tension: 23.1 mN/m),

S-611 (nonionic, C₁₋₆ perfluoroalkyl group-containing polymer, surface tension: 18.4 mN/m),

S-651 (nonionic, C₁₋₆ perfluoroalkyl group-containing polymer, surface tension: 23.0 mN/m),

S-650 (nonionic, C₁₋₆ perfluoroalkyl group-containing polymer), etc.

Manufactured by DIC Corporation, MEGAFACE series:

F-444 (nonionic, perfluoroalkyl ethylene oxide adduct, surface tension: 16.8 mN/m), etc.

Manufactured by Asahi Glass Company, Limited, AsahiGuard series: E100, etc.

### <Preferred Fluorinated Polymer (A)>

As the fluorinated polymer (A), fluorinated polymers (A1) to (A3) as described below are preferred from such a viewpoint that it is thereby possible to easily form a coating layer which is excellent in water resistance, from which coating components are less likely to be eluted, on which protein is less likely to be adsorbed, and which is excellent in biocompatibility. The fluorinated polymers (A1) and (A2) are fluorinated polymers (A) having biocompatible groups only in side chains, and the fluorinated polymer (A3) is a fluorinated polymer (A) having biocompatible groups in at least the main chain.

### «Fluorinated Polymer (A1)»

The fluorinated polymer (A1) is a fluorinated polymer having units (hereinafter referred to also as units (m1)) derived from the following monomer (m1) and at least one member selected from the group consisting of units (hereinafter referred to also as units (m2)) derived from the monomer (m2) and units (hereinafter referred to also as unit (m3)) derived from the monomer (m3).

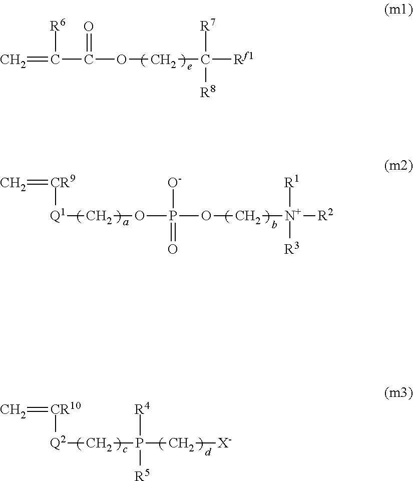

Here, R⁶ is a hydrogen atom, a chlorine atom or a methyl group, e is an integer of from 0 to 3, R⁷ and R⁸ are each independently a hydrogen atom, a fluorine atom or a trifluoromethyl group, R^{f1} is a C₁₋₂₀ perfluoroalkyl group, R⁹ is a hydrogen atom, a chlorine atom or a methyl group, Q¹ is —C(═O)—O— or —C(═O)—NH—, R¹ to R³ are each independently a C₁₋₅ alkyl group, “a” is an integer of from 1 to 5, b is an integer of from 1 to 5, R¹⁰ is a hydrogen atom, a chlorine atom or a methyl group, Q² is —C(═O)—O— or —C(═O)—NH—, R⁴ and R⁵ are each independently a C₁₋₅ alkyl group, X⁻ is the group (3-1) or the group (3-2), c is an integer of from 1 to 20, d is an integer of from 1 to 5.

### Monomer (m1):

In the formula (m1), R⁶ is preferably a hydrogen atom or a methyl group from the viewpoint of polymerization efficiency.

e is, from the viewpoint of excellent flexibility of the fluorinated polymer (A1), preferably an integer of from 1 to 3, particularly preferably 1 or 2. R⁷ and R⁸ are, from the viewpoint of excellent water resistance, each preferably a fluorine atom. The perfluoroalkyl group for R^{f1} may be linear or branched. As R^{f1}, from the viewpoint of easy availability of raw material, a C₁₋₁₀ perfluoroalkyl group is preferred, and a C₁₋₅ perfluoroalkyl group is particularly preferred.

Specific examples of the monomer (m1) may, for example, be the following compounds.

CH₂═C(CH₃)COO(CH₂)₂(CF₂)₅CF₃,

CH₂═CHCOO(CH₂)₂(CF₂)₅CF₃,

CH₂═C(CH₃)COOCH₂CF₃,

CH₂═CHCOOCH₂CF₃,

CH₂═CR₆COO(CH₂)ₑCF₂CF₂CF₃,

CH₂═CR₆COO(CH₂)ₑCF₂CF(CF₃)₂,

CH₂═CR₆COOCH(CF₃)₂,

CH₂═CR₆COOC(CF₃)₃, etc.

As the monomer (m1), from the viewpoint of excellent water resistance, CH₂═C(CH₃)COO(CH₂)₂(CF₂)₅CF₃, CH₂═CHCOO(CH₂)₂(CF₂)₅CF₃ or CH₂═CCH₃COOCH₂CF₃ is particularly preferred. Units (m1) may be of one type, or of two or more types.

### Monomer (m2):

The monomer (m2) is a monomer having a group (2).

In the formula (m2), R⁹ is preferably a hydrogen atom or a methyl group from the viewpoint of polymerization efficiency.

Q¹ is —C(═O)—O— or —C(═O)—NH—, and from such a viewpoint that protein is less likely to be adsorbed, —C(═O)—O— is preferred. R¹ to R³ are each independently a C₁₋₅ alkyl group, and from such a viewpoint that protein is less likely to be adsorbed, a C₁₋₄ alkyl group is preferred, and a methyl group is particularly preferred. “a” is an integer of from 1 to 5, and from the viewpoint of excellent flexibility of the fluorinated polymer (A1), it is preferably an integer of from 1 to 4, particularly preferably 2. b is an integer of from 1 to 5, and from such a viewpoint that protein is less likely to be adsorbed, it is preferably an integer of from 1 to 4, particularly preferably 2.

Specific examples of the monomer (m2) may, for example, be 2-methacryloyloxyethyl phosphorylcholine, 2-acryloyloxyethyl phosphorylcholine, etc.

In a case where the fluorinated polymer (A1) has units (m2), the units (m2) may be of one type, or of two or more types.

### Monomer (m3):

The monomer (m3) is a monomer having a group (3).

In the formula (m3), R¹⁰ is preferably a hydrogen atom or a methyl group from the viewpoint of polymerization efficiency.

Q² is —C(═O)—O— or —C(═O)—NH—, and from such a viewpoint that protein is less likely to be adsorbed on the fluorinated polymer (A1), —C(═O)—O— is preferred. R⁴ and R⁵ are each independently a C₁₋₅ alkyl group, and from the viewpoint of easy availability of raw material, a C₁₋₄ alkyl group is preferred, and a methyl group is particularly preferred. X⁻ is preferably the group (3-1) or the group (3-2). c is an integer of from 1 to 20, and from the viewpoint of easy availability of raw material, it is preferably an integer of from 1 to 15, more preferably an integer of from 1 to 10, particularly preferably 2. d is an integer of from 1 to 5, and from such a viewpoint that protein is less likely to be adsorbed, it is preferably an integer of from 1 to 4, particularly preferably 1.

Specific examples of the monomer (m3) may, for example, be the following compounds.
N-Methacryloyloxyethyl-N,N-dimethylammonium-α-N-methylcarboxy betaine,
N-acryloyloxyethyl-N,N-dimethylammonium-α-N-methylcarboxy betaine,
N-methacryloyloxyethyl-N,N-dimethylammonium-α-N-propylsulfoxy betaine,
N-methacryloylaminopropyl-N,N-dimethylammonium-α-N-propylsulfoxy betaine, etc.

As the monomer (m3), from such a viewpoint that protein is less likely to be adsorbed, N-methacryloyloxyethyl-N,N-dimethylammonium-α-N-methylcarboxy betaine or N-acryloyloxyethyl-N,N-dimethylammonium-α-N-methylcarboxy betaine is preferred. In a case where the fluorinated polymer (A1) has units (m3), the units (m3) may be of one type, or of two or more types.

From such a viewpoint that protein is less likely to be adsorbed, it is particularly preferred that the fluorinated polymer (A1) has, as units having a biocompatible group, either one of units (m2) or units (m3). Here, the fluorinated polymer (A1) may have all of units (m1), units (m2) and units (m3).

The fluorinated polymer (A1) may have, in addition to units (m1) and at least one member selected from the group consisting of units (m2) and units (m3), units derived from another monomer other than for units (1), units (m2) and units (m3).

As such another monomer, from the viewpoint of excellent water resistance, the following monomer (m7) is preferred.

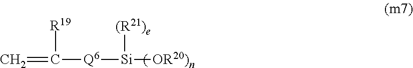

Here, R¹⁹ is a hydrogen atom, a chlorine atom or a methyl group, Q⁶ is a 6-membered aromatic hydrocarbon group (—C₆H₄—) or —C(═O)O—(CH₂)_{ρ}— (wherein ρ is an integer of from 1 to 100), and R²⁰ and R²¹ are each independently a C₁₋₃ alkyl group. n is an integer of from 1 to 3, and η+ƒ is 3.

In the formula (m7), R¹⁹ is, from the viewpoint of polymerization efficiency, preferably a hydrogen atom or a methyl group.

Q⁶ is, from the viewpoint of easy availability, preferably —C(═O)O—(CH₂)₂—. R²⁰ and R²¹ are, from the viewpoint of easy availability, each independently, preferably a C₁₋₃ alkyl group, particularly preferably a C₁₋₂ alkyl group. η is, from the viewpoint of adhesion to a substrate, preferably 2 or 3.

Specific examples of the monomer (m7) may, for example, be p-styryl trimethoxysilane, 3-methacryloyloxypropyltrimethoxysilane, 3-methacryloyloxypropylmethyldimethoxysilane, 3-methacryloyloxypropylmethyldiethoxysilane, 3-methacryloyloxypropyltriethoxysilane, 3-acryloyloxypropyltrimethoxysilane, etc.

As the monomer (m7), 3-methacryloyloxypropyltrimethoxysilane, 3-methacryloxypropylmethyldimethoxysilane, 3-methacryloxypropylmethyldiethoxysilane, 3-methacryloxypropyltriethoxysilane or 3-acryloxypropyltrimethoxysilane is preferred.

In a case where the fluorinated polymer (A1) has units (m7) derived from a monomer (m7), the units (m7) may be of one type, or of two or more types.

As other monomer other than the monomer (m7), for example, compounds listed as other monomers in the fluorinated polymer (A1) may be mentioned.

Such other monomers other than the monomer (m7) may, for example, be N,N-dimethylaminoethyl (meth)acrylate, N,N-diethylaminoethyl (meth)acrylate, N-(meth)acryloylmorpholine, N-(meth)acryloylpyridine, N,N-dimethylamino-oxide ethyl (meth)acrylate, N,N-diethylamino-oxide ethyl (meth)acrylate, etc. Further, 2-isocyanatoethyl (meth)acrylate, 3,5-dimethylpyrazole adduct of 2-isocyanatoethyl (meth)acrylate, 3-isocyanatepropyl (meth)acrylate, 4-isocyanatebutyl (meth)acrylate, triallyl isocyanurate, glycidyl (meth)acrylate, a polyoxyalkylene glycol monoglycidyl ether (meth)acrylate may also be used.

The proportion of units (m1) to all units in the fluorinated polymer (A1) is preferably from 5 to 95 mol %, particularly preferably from 10 to 90 mol %. When the proportion of units (m1) is at least the above lower limit value, water resistance will be excellent, and when it is at most the above upper limit value, protein will be less likely to be adsorbed.

The proportion of units having a biocompatible group to all units in the fluorinated polymer (A1) is preferably from 5 to 95 mol %, particularly preferably from 10 to 90 mol %. When the proportion of the units is at least the above lower limit value, protein will be less likely to be adsorbed, and when it is at most the above upper limit value, water resistance will be excellent.

The total proportion of units (m2) and units (m3) to all units in the fluorinated polymer (A1) is preferably from 5 to 95 mol %, particularly preferably from 10 to 90 mol %. When the total proportion of units (m2) and units (m3) is at least the above lower limit value, protein will be less likely to be adsorbed, and when it is at most the above upper limit value, water resistance will be excellent.

In a case where the fluorinated polymer (A1) has units (m7), the proportion of units (m7) to all units in the fluorinated polymer (A1) is preferably from 0.1 to 10 mol %, particularly preferably from 0.5 to 10 mol %. When the proportion of units (m7) is at least the above lower limit value, water resistance will be excellent, and when it is at most the above upper limit value, protein will be less likely to be adsorbed.

The fluorinated polymer (A1) is obtainable by carrying out a polymerization reaction of the monomers in a polymerization solvent by using a known method.

The polymerization solvent is not particularly limited, and may, for example, be a ketone (acetone, methyl ethyl ketone, methyl isobutyl ketone, etc.), an alcohol (methanol, 2-propyl alcohol, etc.), an ester (ethyl acetate, butyl acetate, etc.), an ether (diisopropyl ether, tetrahydrofuran, dioxane, etc.), a glycol ether (ethyl ether or methyl ether of ethylene glycol, propylene glycol or dipropylene glycol, etc.) and its derivatives, an aliphatic hydrocarbon, an aromatic hydrocarbon, a halogenated hydrocarbon (perchloroethylene, trichloro-1,1,1-ethane, trichlorotrifluoroethane, dichloropentafluoropropane, etc.), dimethylformamide, N-methyl-2-pyrrolidone, butyloacetone, dimethyl sulfoxide (DMSO), etc.

The total concentration of all monomers in the reaction solution in the polymerization reaction for obtaining the fluorinated polymer (A1) is preferably from 5 to 60 mass %, particularly preferably from 10 to 40 mass %.

In the polymerization reaction for obtaining the fluorinated copolymer (A1), it is preferred to use a polymerization initiator. The polymerization initiator may, for example, be a peroxide (benzyl peroxide, lauryl peroxide, succinyl peroxide, tert-butyl perpivalate, etc.), an azo compound, etc.

As the polymerization initiator, 2,2′-azoisobutyronitrile, 2,2′-azobis-2-methylbutyronitrile, dimethyl-2,2′-azobis isobutyrate, 2,2′-azobis[2-(2-imidazolin-2-yl)propane], 2,2′-azobis(4-methoxy-2,4-dimethylvaleronitrile), 1,1′-azobis(2-cyclohexane-1-carbonitrile), 2,2′-azobis(2,4-dimethylvaleronitrile), 1,1′-azobis(1-acetoxy-1-phenylethane), dimethylazobisisobutyrate, or 4,4′-azobis(4-cyanovaleric acid) is preferred, and 2,2′-azoisobutyronitrile, 2,2′-azobis[2-(2-imidazolin-2-yl)propane] or 4,4′-azobis(4-cyanovaleric acid) is particularly preferred.

The amount of the polymerization initiator is preferably from 0.1 to 1.5 parts by mass, more preferably from 0.1 to 1.0 part by mass, to the total amount of 100 parts by mass of monomers.

In order to adjust the polymerization degree (molecular weight) of the fluorinated polymer (A1), a chain transfer agent may be used in the polymerization reaction. By using a chain transfer agent, there will also be an effect to increase the total concentration of monomers in the polymerization solvent.

The chain transfer agent may, for example, be an alkyl mercaptan (tert-dodecyl mercaptan, n-dodecyl mercaptan, stearyl mercaptan, etc.), aminoethanethiol, mercaptoethanol, 3-mercaptopropionic acid, 2-mercaptopropionic acid, thiomalic acid, thioglycolic acid, 3,3′-dithio-dipropionate, 2-ethylhexyl thioglycolate, n-butyl thioglycolate, methoxybutyl thioglycolate, ethyl thioglycolate, 2,4-diphenyl-4-methyl-1-pentene, carbon tetrachloride, etc.

The amount of the chain transfer agent is preferably from 0 to 2 parts by mass, more preferably from 0.1 to 1.5 parts by mass, to the total amount of 100 parts by mass of monomers.

The reaction temperature in the polymerization reaction is preferably within a range of from room temperature to the boiling point of the reaction solution. From the viewpoint of efficiently using the polymerization initiator, at least the half-life temperature of the polymerization initiator is preferred, from 30 to 90° C. is more preferred, and from 40 to 80° C. is further preferred.

### «Fluorinated Polymer (A2)»

The fluorinated polymer (A2) is a fluorinated polymer having units (m1) derived from the above monomer (m1) and units (hereinafter referred to also as units (m4)) derived from the following monomer (m4).

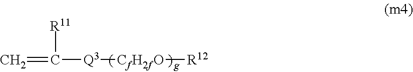

Here, in the formula, R¹¹ is a hydrogen atom, a chlorine atom or a methyl group, Q³ is —COO— or —COO(CH₂)ₕ—NHCOO— (wherein h is an integer of from 1 to 4), R¹² is a hydrogen atom or —(CH₂)ᵢ—R¹³ (wherein R¹³ is a C₁₋₈ alkoxy group, a hydrogen atom, a fluorine atom, a trifluoromethyl group or a cyano group, and i is an integer of from 1 to 25), f is an integer of from 1 to 10, and g is an integer of from 1 to 100.

### Monomer (m1):

The preferred range and examples of the monomer (m1) are the same as those described in the fluorinated polymer (A1). Units (m1) may be of one type, or of two or more types.

Monomer (m4): The monomer (m4) is a monomer having a group (1). In the formula (m4), R¹¹ is, from the viewpoint of polymerization efficiency, preferably a hydrogen atom or a methyl group, particularly preferably a methyl group. Q³ is preferably —COO—. R¹² is preferably a hydrogen atom.

In a case where g is 2 or more, the plurality of (C_{f}H_{2f}O) may be the same or different. If different, their disposition may be any of random, block and alternating (e.g. (CH₂CH₂O—CH₂CH₂CH₂CH₂O), etc.). If f is 3 or more, C_{f}H_{2f}O may have a linear structure or a branched structure. (C_{f}H_{2f}O) may, for example, be (CH₂O), (CH₂CH₂O), (CH₂CH₂CH₂O), (CH(CH₃)CH₂O), (CH₂CH₂CH₂CH₂O), etc. f is preferably an integer of from 1 to 6, particularly preferably an integer of from 1 to 4, from such a viewpoint that protein is less likely to be adsorbed. g is preferably an integer of from 1 to 50, more preferably an integer of from 1 to 30, particularly preferably an integer of from 1 to 20, from such a viewpoint that an excluded volume effect is high and protein is less likely to be adsorbed. i is preferably an integer of from 1 to 4, particularly preferably 1 or 2, from the viewpoint of excellent flexibility of the fluorinated polymer (A2).

R¹³ is preferably an alkoxy group from such a viewpoint that protein is less likely to be adsorbed.

As the monomer (m4), a monomer (m41) represented by the following formula (m41) is preferred.

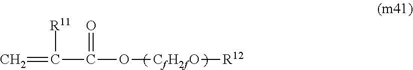

Specific examples of the monomer (m4) may, for example, be the following compounds.

CH₂═CH—COO—(C₂H₄O)₉—H,

CH₂═CH—COO—(C₂H₄O)₄—H,

CH₂═CH—COO—(C₂H₄O)₅—H,

CH₂═CH—COO—(C₂H₄O)₉—CH₃,

CH₂═C(CH₃)—COO—(C₂H₄O)₉—H,

CH₂═C(CH₃)—COO—(C₂H₄O)₄—H,

CH₂═C(CH₃)—COO—(C₂H₄O)₅—H,

CH₂═C(CH₃)—COO—(C₂H₄O)₉—CH₃,

CH₂═CH—COO—(CH₂O)—(C₂H₄O)_{g1}—CH₂—OH,

CH₂═CH—COO—(C₂H₄O)_{g2}—(C₄H₈O)_{g3}—H,

CH₂═C(CH₃)—COO—(C₂H₄O)_{g2}—(C₄H₈O)_{g3}—H,

CH₂═CH—COO—(C₂H₄O)_{g2}—(C₄H₈O)_{g3}—CH₃,

CH₂═C(CH₃)—COO—(C₂H₄O)_{g2}—(C₄H₈O)_{g3}—CH₃, etc.

In the above formulae, g1 is an integer of from 1 to 20, and g2 and g3 are each independently an integer of from 1 to 50.

As the monomer (m4), from such a viewpoint that protein is less likely to be adsorbed, the following compounds are preferred.

CH₂═CH—COO—(C₂H₄O)₉—H,

CH₂═CH—COO—(C₂H₄O)₄—H,

CH₂═CH—COO—(C₂H₄O)₅—H,

CH₂═C(CH₃)—COO—(C₂H₄O)₉—CH₃,

CH₂═CH—COO—(CH₂O)—(C₂H₄O)_{g1}—CH₂—OH,

CH₂═C(CH₃)—COO—(C₂H₄O)_{g2}—(C₄H₈O)_{g3}—H.

The fluorinated polymer (A2) may have units derived from another monomer other than the monomer (m1) and the monomer (m4).

As such another monomer, from the viewpoint of excellent water resistance, a monomer (m5) represented by the following formula (m5) is preferred.

CH₂═CR¹⁴—COO-Q⁴-R¹⁵ (m5)

Here, R¹⁴ is a hydrogen atom, a chlorine atom or a methyl group, R¹⁵ is a C₁₋₈ alkoxy group, a hydrogen atom, a hydroxy group or a cyano group, and Q⁴ is a single bond, a C₁₋₂₀ alkylene group, a C₁₋₁₂ polyfluoroalkylene group or —CF₂—(OCF₂CF₂)_{y}—OCF₂— (wherein y is an integer of from 1 to 6).

In the formula (m5), R¹⁴ is, from the viewpoint of polymerization efficiency, preferably a hydrogen atom or a methyl group, particularly preferably a hydrogen atom. y is, from the viewpoint of excellent flexibility of the fluorinated polymer (A2), preferably an integer of from 1 to 15, particularly preferably an integer of from 2 to 15. The alkylene group and polyfluoroalkylene group for Q⁴ may be linear or branched. Q⁴ is, from the viewpoint of excellent flexibility of the fluorinated polymer (A2), preferably a C₁₋₁₂ alkylene group, particularly preferably a methylene group or an isobutylene group. R¹⁵ is, from the viewpoint of excellent water resistance, preferably a hydrogen atom. Specific examples of the monomer (m5) may, for example, be the following compounds.

CH₂═CH—COO—(CH₂)₄—H,

CH₂═CH—COO—(CH₂)₆—H,

CH₂═CH—COO—(CH₂)₈—H,

CH₂═CH—COO—(CH₂)₁₆—H,

CH₂═CH—COO—CH₂CH(C₂H₅)CH₂CH₂CH₂CH₃, etc.

As the monomer (m5), CH₂═CH—COO—(CH₂)₄—H, CH₂═CH—COO(CH₂)₈—H or CH₂═CH—COO—(CH₂)₁₆—H is preferred, and CH₂═CH—COO—(CH₂)₈—H or CH₂═CH—COO—(CH₂)₁₆—H is particularly preferred.

From the viewpoint of excellent water resistance, it is also preferred that the fluorinated polymer (A2) has units (m7) derived from a monomer (m7). The preferred embodiment of the monomer (m7) is the same as in the case of the fluorinated polymer (A1).

Further, as another monomer other than the monomer (m5) and the monomer (m7), the same compound as the compound mentioned as another monomer other than the monomer (m7) in the fluorinated polymer (A1) may be mentioned.

In a case where the fluorinated polymer (A2) has units (m5), the units (m5) may be of one type, or of two or more types.

In a case where the fluorinated polymer (A2) has units (m5) in addition to units (m1) and units (m4), particularly preferred is a fluorinated polymer having CH₂═CHCOO(CH₂)₂(CF₂)₅CF₃ units, CH₂═CH—COO—(CH₂O)—(C₂H₄O)_{g1}—CH₂—OH (g1=1 to 20) units, and CH₂═CH—COO—(CH₂)₁₆—H units.

The proportion of units (m1) to all units in the fluorinated polymer (A2) is preferably from 5 to 95 mol %, particularly preferably from 10 to 90 mol %. When the proportion of units (m1) is at least the above lower limit value, water resistance will be excellent, and when it is at most the above upper limit value, protein will be less likely to be adsorbed.

The proportion of units (m4) to all units in the fluorinated polymer (A2) is preferably from 5 to 95 mol %, particularly preferably from 10 to 90 mol %. When the proportion of units (m4) is at least the above lower limit value, protein will be less likely to be adsorbed, and when it is at most the above upper limit value, water resistance will be excellent.

In a case where the fluorinated polymer (A2) has units (m5), the proportion of units (m5) to the total of units (m1) and units (m4) is preferably from 5 to 95 mol %, particularly preferably from 10 to 90 mol %. When the proportion of units (m5) is at least the above lower limit value, water resistance will be excellent, and when it is at most the above upper limit value, protein will be less likely to be adsorbed.

In a case where the fluorinated polymer (A2) has units (m7), the proportion of units (m7) to all units in the fluorinated polymer (A2) is preferably from 0.1 to 10 mol %, particularly preferably from 0.5 to 10 mol %. When the proportion of units (m7) is at least the above lower limit value, water resistance will be excellent, and when it is at most the above upper limit value, protein will be less likely to be adsorbed.

The fluorinated polymer (A2) may be produced in the same manner as the fluorinated polymer (A1) except that the monomers (m1), (m4), (m5) and (m7) are used.

### «Fluorinated Polymer (A3)»

The fluorinated polymer (A3) is a block copolymer having a segment (I) comprising units (hereinafter referred to also as units (m6)) derived from a monomer (m6) represented by the following formula (m6) and a segment (II) comprising a molecular chain derived from a polymeric azo initiator having a structure (hereinafter referred to also as the structure (6)) represented by the following formula (6). The molecular chain of the structure (6) is made of a unit having a group (1) as a biocompatible group. Thus, the fluorinated polymer (A3) has groups (1) in the main chain.

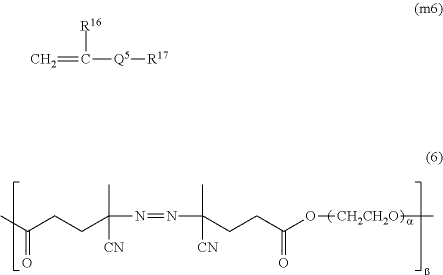

Here, in the above formulae, R¹⁶ is a hydrogen atom, a chlorine atom or a methyl group, Q⁵ is a single bond or a divalent organic group, R¹⁷ is a C₁₋₆ polyfluoroalkyl group which may have an etheric oxygen atom between carbon-carbon atoms, α is an integer of from 5 to 300, and β is an integer of from 1 to 20.

### Segment (I):

The segment (I) is a segment comprising a molecular chain having units (m6).

In the formula (m6), R¹⁶ is a hydrogen atom, a C₁₋₄ alkyl group or a halogen atom, and from the viewpoint of easy availability of raw material, a hydrogen atom or a methyl group is preferred.

Q⁵ may, for example, be the following groups from the viewpoint of efficiency in synthesis and the physical properties of the fluorinated polymer (A3).

—O—, —S—, —NH—, —SO₂—, —PO₂—, —CH═CH—, —CH═N—, —N═N—, —N(O)═N—, —OCO—, —COO—, —COS—, —CONH—, —COCH₂—, —CH₂CH₂—, —CH₂—, —CH₂NH—, —CO—, —CH═CH—COO—, —CH═CH—CO—, a linear or branched alkylene group, an alkenylene group, an alkyleneoxy group, a divalent 4- to 7-membered ring substituent, a divalent 6-membered aromatic hydrocarbon group, a divalent 4- to 6-membered alicyclic hydrocarbon group, a divalent 5- or 6-membered heterocyclic group, a condensed ring thereof, a group constituted by a combination of divalent linking groups, etc.

A divalent organic group may have a substituent. The substituent may, for example, be a hydroxy group, a halogen atom (a fluorine atom, a chlorine atom, a bromine atom or an iodine atom), a cyano group, an alkoxy group (a methoxy group, an ethoxy group, a butoxy group, an octyloxy group, a methoxyethoxy group, etc.), an aryloxy group (a phenoxy group, etc.), an alkylthio group (a methylthio group, an ethylthio group, etc.), an acyl group (an acetyl group, a propionyl group, a benzoyl group, etc.), a sulfonyl group (a methanesulfonyl group, a benzenesulfonyl group), an acyloxy group (an acetoxy group, a benzoyloxy group), a sulfonyloxy group (a methanesulfonyloxy group, a toluenesulfonyloxy group, etc.), a phosphonyl group (a diethylphosphonyl group, etc.), an amide group (an acetylamino group, a benzoylamino group, etc.), a carbamoyl group (an N,N-dimethylcarbamoyl group, an N-phenylcarbamoyl group, etc.), an alkyl group (a methyl group, an ethyl group, a propyl group, an isopropyl group, a cyclopropyl group, a butyl group, a 2-carboxyethyl group, a benzyl group, etc.), an aryl group (a phenyl group, a tolyl group, etc.), a heterocyclic group (a pyridyl group, an imidazolyl group, a furanyl group, etc.), an alkenyl group (a vinyl group, a 1-propenyl group, etc.), an alkoxy acyloxy group (an acetyloxy group, a benzoyloxy group, etc.), an alkoxycarbonyl group (a methoxycarbonyl group, an ethoxycarbonyl group, etc.), a polymerizable group (a vinyl group, an acryloyl group, a methacryloyl group, a styryl group, a cinnamic acid residue, etc.), etc.

As Q⁵, a single bond, —O—, —(CH₂CH₂O)_{γ}— (wherein γ is an integer of from 1 to 10), —COO—, a 6-membered aromatic hydrocarbon group (hereinafter referred to also as “-Benz-”), a linear or branched alkylene group, a linear or branched alkylene group in which part of hydrogen atoms has been substituted by a hydroxy group, or a group constituted by a combination of these bivalent linking groups, is preferred, and a single bond, a C₁₋₅ alkylene group or —COOY¹— is particularly preferred. Y¹ may, for example, be —(CH₂)δ-, —(CH₂)δ-CH(OH)—(CH₂)ε-, —(CH₂)δ-NR¹⁸—SO₂—, etc., and —(CH₂)δ- is particularly preferred. Here, δ is an integer of from 1 to 5, ε is an integer of from 1 to 5, and R¹⁸ is a hydrogen atom or a C₁₋₃ alkyl group.

In a case where Q⁵ is —(CH₂CH₂O)_{γ}—, the fluorinated polymer (A3) has a biocompatible group in both the main chain and side chain.

R¹⁷ is a C₁₋₆ polyfluoroalkyl group which may have an etheric oxygen atom between carbon-carbon atoms. From the viewpoint of excellent water resistance, R¹⁷ is preferably a C₃₋₆ polyfluoroalkyl group, particularly preferably a C₄ or C₆ polyfluoroalkyl group. R¹⁷ may be linear or may be branched. Further, the polyfluoroalkyl group for R¹⁷ is preferably a perfluoroalkyl group from the viewpoint of excellent water resistance.

Specific examples of the monomer (m6) may, for example, be the following corn pounds.

CH₂═CH—COO(CH₂)₂(CF₂)₃CF₃,

CH₂═CH—COO(CH₂)₂(CF₂)₅CF₃,

CH₂═C(CH₃)COO(CH₂)₂(CF₂)₃CF₃,

CH₂═C(CH₃)COO(CH₂)₂(CF₂)₅CF₃,

CH₂═CHCOO(CH₂)₃(CF₂)₃CF₃,

CH₂═CHCOO(CH₂)₃(CF₂)₅CF₃,

CH₂═CHCOOCH₂CH(OH)CH₂(CF₂)₃CF₃,

CH₂═CHCOOCH₂CH(OH)CH₂(CF₂)₅CF₃,

CH₂═C(CH₃)COO(CH₂)₃(CF₂)₃CF₃,

CH₂═C(CH₃)COO(CH₂)₃(CF₂)₅CF₃,

CH₂═C(CH₃)COOCH₂CH(OH)CH₂(CF₂)₃CF₃,

CH₂═C(CH₃)COOCH₂CH(OH)CH₂(CF₂)₅CF₃,

CH₂═CH-benz-(CF₂)₃CF₃,

CH₂═CH-benz-(CF₂)₅CF₃,

CH₂═CHCOOCH₂CH₂N(CH₃)SO₂(CF₂)₃CF₃,

CH₂═CHCOOCH₂CH₂N(CH₃)SO₂(CF₂)₅CF₃,

CH₂═C(CH₃)COOCH₂CH₂N(CH₃)SO₂(CF₂)₃CF₃,

CH₂═C(CH₃)COOCH₂CH₂N(CH₃)SO₂(CF₂)₅CF₃,

CH₂═CHCOOCH₂CH₂N(C₂H₅)SO₂(CF₂)₃CF₃,

CH₂═CHCOOCH₂CH₂N(C₂H₅)SO₂(CF₂)₅CF₃,

CH₂═C(CH₃)COOCH₂CH₂N(C₂H₅)SO₂(CF₂)₃CF₃,

CH₂═C(CH₃)COOCH₂CH₂N(C₂H₅)SO₂(CF₂)₅CF₃,

CH₂═CHCOO(CH₂)₂N(CH₂CH₂CH₃)SO₂(CF₂)₃CF₃,

CH₂═CHCOO(CH₂)₂N(CH₂CH₂CH₃)SO₂(CF₂)₅CF₃,

CH₂═C(CH₃)COO(CH₂)₂N(CH₂CH₂CH₃)SO₂(CF₂)₃CF₃,

CH₂═C(CH₃)COO(CH₂)₂N(CH₂CH₂CH₃)SO₂(CF₂)₅CF₃,

CH₂═CHCONHCH₂C₄F₉,

CH₂═CHCONHCH₂C₅F₁₁,

CH₂═CHCONHCH₂C₆F₁₃,

CH₂═CHCONHCH₂CH₂OCOC₄F₉,

CH₂═CHCONHCH₂CH₂OCOC₅F₁₁,

CH₂═CHCONHCH₂CH₂OCOC₆F₁₃,

CH₂═CHCOOCH(CF₃)₂,

CH₂═C(CH₃)COOCH(CF₃)₂, etc.

To all units of the fluorinated polymer (A3), the proportion of units (m6) is preferably from 1 to 99 mol %, particularly preferably from 1 to 90 mol %. When the proportion of units (m6) is at least the above lower limit value, water resistance will be excellent. When the proportion of units (m6) is at most the above upper limit value, protein will be less likely to be adsorbed.

The proportion of units (m6) in the segment (I) (100 mass %) is preferably from 5 to 100 mass %, particularly preferably from 10 to 100 mass %. When the proportion of units (m6) is at least the lower limit value in the above range, polymerization of the monomers to constitute the segment (I) will be facilitated.

### Segment (II):

The segment (II) is a segment comprising a molecular chain derived from a polymeric azo initiator having the structure (6).

α in the formula (6) is an integer of from 5 to 300, and from such a viewpoint that protein is less likely to be adsorbed, it is preferably an integer of from 10 to 200, particularly preferably an integer of from 20 to 100.

β is an integer of from 1 to 20, and from the viewpoint of polymerization efficiency, it is preferably an integer of from 2 to 20, particularly preferably an integer of from 5 to 15.

The polymeric azo initiator having the structure (6) may, for example, be VPE series (VPE-0201, VPE-0401, VPE-0601) manufactured by Wako Pure Chemical Industries, Ltd., etc.

To all units in the fluorinated polymer (A3), the total proportion of the respective units in the molecular chain of the structure (6) is preferably from 1 to 50 mol %, particularly preferably from 1 to 40 mol %. When the proportion of the units is at least the above lower limit value, protein will be less likely to be adsorbed. When the proportion is at most the above upper limit value, water resistance will be excellent.

The fluorinated polymer (A3) can be produced by the same method as for the fluorinated polymer (A1) except that the monomer (m6) and the polymeric azo initiator having the structure (6) are used. In the polymerization reaction for obtaining the fluorinated polymer (A3), as a polymerization initiator, in addition to the polymeric azo initiator having the structure (6), the polymerization initiator mentioned in the case of the fluorinated polymer (A1) may be used in combination.

In the present invention, as the fluorinated polymer (A), only one of the fluorinated polymers (A1) to (A3) may be used, or two or more selected from the group consisting of fluorinated polymers (A1) to (A3) may be used in combination.

Further, the fluorinated polymer (A) is not limited to the above-described fluorinated polymers (A1) to (A3).

### (Method for Forming Layer Formed of Fluorinated Polymer)

When the fluorinated polymer (A) of the present invention is liquid at room temperature (from 20 to 25° C.), it may be applied to the substrate as it is. Otherwise, as the case requires, a coating solution containing a solvent (hereinafter referred to also as “solvent (B)” in addition to the fluorinated polymer (A) may be applied to a substrate, and then the solvent is removed to form a layer formed of the fluorinated polymer (A).

At the time of applying the coating solution, components other than the fluorinated polymer (A) and solvent (B), e.g. a leveling agent, a crosslinking agent, etc., may be incorporated in the coating solution for application. In a case where no crosslinking agent is incorporated in the coating solution, the layer to be formed will be a layer formed of only the fluorinated polymer (A). Whereas, in a case where a crosslinking agent is incorporated in the coating solution, the layer to be formed will be a layer formed from the fluorinated polymer (A) and the crosslinking agent.

The solvent (B) may, for example, be a non-fluorinated solvent, a fluorinated solvent, etc., and the non-fluorinated solvent may, for example, be an alcohol solvent, a halogen-containing solvent, etc. For example, ethanol, methanol, acetone, chloroform, ASAHIKLIN AK225 (manufactured by Asahi Glass Company, Limited), AC6000 (manufactured by Asahi Glass Company, Limited), etc. may be mentioned. As the solvent (B), it is preferred to select the type that does not dissolve the substrate. In the case of using polystyrene as the material for the substrate, ethanol, methanol, ASAHIKLIN AK225 (manufactured by Asahi Glass Company, Limited), AC6000 (manufactured by Asahi Glass Company, Limited), etc. are preferred.

The concentration of the fluorinated polymer (A) in the coating solution is preferably from 0.0001 to 10 mass %, particularly preferably from 0.0005 to 5 mass %. When the concentration of the fluorinated polymer (A) is within the above range, it is possible to uniformly apply the coating solution thereby to form a uniform coating layer.

The coating solution may contain other components other than the fluorinated polymer (A) and solvent (B), as the case requires. Other components may, for example, be a leveling agent, a crosslinking agent, etc.

In a case where a cell-trapping filter is to be used for a long time, by adding to the coating solution a crosslinking agent capable of crosslinking the fluorinated polymer (A) thereby to adjust the degree of crosslinking in the coating layer, it is possible to form a coating layer having excellent durability whereby excellent biocompatibility can be maintained over a longer period of time. Specifically, when the fluorinated polymer (A) has a hydroxy group, by adding a crosslinking agent which reacts with the hydroxy group, it is possible to form a coating layer having excellent durability. Particularly in the case of using a fluorinated polymer comprising units having a hydroxy group (e.g. a fluorinated polymer (A2) comprising units (m4) wherein R¹² is a hydrogen atom), it is preferred to add a crosslinking agent that reacts with the hydroxy group.

As the crosslinking agent which reacts with a hydroxy group, a polyfunctional isocyanate compound may be mentioned. The polyfunctional isocyanate compound may, for example, be hexamethylene diisocyanate (HDI), a HDI-type polyisocyanate, isophorone diisocyanate (IPDI), etc. The HDI-type polyisocyanate may, for example, be a biuret type for the two-liquid type, an isocyanurate type, an adduct type, a bifunctional type, etc., and also includes a block type having a threshold value in the curing initiation temperature. As the HDI-type polyisocyanate, a commercially available product may be used, and Duranate (manufactured by Asahi Kasei Corporation), etc. may be mentioned.

The polyfunctional isocyanate compound to be used, may be suitably selected for use depending upon the reaction temperature, the material for the substrate. For example, in a case where polystyrene is used as the material for the substrate, a biuret type, an isocyanurate type or the like is preferred, which can be dissolved in ASAHIKLIN AK225 (manufactured by Asahi Glass Company, Limited), AC6000 (manufactured by Asahi Glass Company, Limited), etc., and whereby a curing reaction proceeds even at a temperature of not higher than 80° C. as the heat distortion temperature of polystyrene.

The degree of crosslinking in the coating layer is determined by the amount of hydroxy groups in the fluorinated polymer (A), the amount of the crosslinking agent to be added and the reaction rate, and may be suitably adjusted within a range not to impair the effects of the present invention.

The amount of the crosslinking agent is preferably from 0.01 to 10 parts by mass, particularly preferably from 0.1 to 1 part by mass, per 100 parts by mass of the fluorinated polymer (A). When the amount of the crosslinking agent is at least the lower limit value in the above range, it is easy to form a coating layer excellent in durability. When the amount of the crosslinking agent is at most the upper limit value in the above range, it is easy to form a coating layer excellent in the biocompatibility.

As described above, the layer formed of the fluorinated polymer of the present invention contains the fluorinated polymer (A) having biocompatible groups and having the proportion P controlled to be within a specific range, whereby it is excellent in water resistance, coating components are less likely to be eluted from the layer, and protein is less likely to be adsorbed.

### (Substrate)

In the present invention, the material of the substrate is not particularly limited. As preferred specific examples, resins such as an ethylene/tetrafluoroethylene copolymer (ETFE), polycarbonate (PC), polyethylene naphthalate (PEN), polyethersulfone (PES), polyethylene terephthalate (PET), polystyrene (PS), polytetrafluoroethylene (PTFE), a cycloolefin polymer (COP), an ethylene/vinyl acetate copolymer (EVA), an ethylene/vinyl alcohol copolymer (EVOH), a tetrafluoroethylene/hexafluoropropylene copolymer (FEP), a high density polyethylene (HDPE), a low density polyethylene (LDPE), a biaxially oriented polypropylene (OPP), polyimide (PA), polyamideimide (PAI), an ethylene/chlorotrifluoroethylene copolymer (ECTFE), polyethylene (PE), polyether ether ketone (PEEK), polyimide (PI), polymethyl methacrylate (PMMA), polypropylene (PP), polyvinyl alcohol (PVA) and polyvinylidene fluoride (PVDF) may be mentioned.

Further, inorganic glass such as quartz glass, borosilicate glass, soda lime glass, alkali-free glass, alkali glass and aluminosilicate glass may be mentioned.

Usually, the cell-trapping filter is disposable, preferred is a resin with low material cost and processing cost. On the other hand, for higher precision analysis, for example, single cell analysis, preferred is inorganic glass which has high transparency of the material itself, which emits less fluorescence, and which is chemically stable and is excellent in rigidity.

The shape of the substrate is usually a sheet shape or a film shape but is not particularly limited. The thickness of the substrate is usually from 5 μm to 1 mm from so that the substrate withstands the pressure applied when the cell fluid passes through the substrate, but it varies depending upon the material of the substrate. In a case where the substrate is made of a resin, the thickness is preferably from 3 μm to 200 μm, more preferably from 5 μm to 25 μm. Further, when the substrate is made of glass, the thickness is preferably from 50 μm to 2 mm, more preferably from 80 μm to 1 mm.

As the coating layer formed on the surface of the substrate, a layer formed only of the fluorinated polymer (A) and a layer formed of the fluorinated polymer (A) and the crosslinking agent may be mentioned.

The thickness of the coating layer is preferably from 1 nm to 1 mm, particularly preferably from 5 nm to 800 μm. When the thickness of the coating layer is at least the above lower limit value, protein is less likely to be adsorbed, and when it is at most the above upper limit value, the coating layer is likely to adhere to the surface of the substrate.

In order to improve adhesion between the coating layer and the substrate, an adhesive layer may be provided between the substrate and the coating layer. As an adhesive to form the adhesive layer, an adhesive having sufficient adhesion to both the substrate and the coating layer may properly be used. For example, a cyanoacrylate adhesive, a silicone-modified acrylic adhesive or an epoxy-modified silicone adhesive, which is an adhesive for an fluororesin, may be mentioned, although a proper adhesive varies depending on the material of the substrate.

As a specific example, for example, in a case where the substrate is made of polystyrene, a cyanoacrylate adhesive is used. In such a case, on the substrate side of the adhesive layer, a cyanoacrylate monomer in the cyanoacrylate adhesive reacts with moisture in the air or on the surface of the substrate and is cured. Since biocompatible groups derived from the fluorinated polymer (A) are present in the coating layer, moisture is present in the coating layer and a periphery thereof. Accordingly, even on the coating layer side of the adhesive layer, the cyanoacrylate monomer reacts with such moisture and is cured. By such an adhesive layer, adhesion between the coating layer and the substrate can be improved.

The cell-trapping filter of the present invention comprises a cell-separating mechanism by size, on the substrate. As the cell-separating mechanism, through-holes which pierce the substrate from the front side to the rear side, or a pillar-like structure may, for example, be mentioned.

The (transverse) cross sectional shape of the through-holes is preferably circular for an application in which cells are arranged while single cells are trapped, and for an application in which rare cells are effectively trapped with a small area, the shape may be rectangular, triangular, square or elliptic. Further, the shape (of the longitudinal cross section) of the through-holes as observed from the substrate thickness direction may be straight, gradually thinning shape, gradually thickening shape, concave or a circular truncated cone shape.

In a case where the cell-separating mechanism is through-holes, the average diameter of the (transverse) cross section is determined by the size of cells to be trapped, and is usually preferably from 500 nm to 100 μm, more preferably from 600 nm to 30 μm. In a case where rare cells such as circulating tumor cells in blood are to be trapped, the average diameter of the cross section of the through-holes is preferably from 4 to 12 μm, more preferably from 4 to 10 μm. Within such a range, cells in the blood pass through the through-holes, and rare cells can effectively be trapped on the substrate.

In the case of through-holes of which the (transverse) cross sectional shape is rectangular or elliptic, formed on the substrate made of a resin film, the short width is preferably from 0.5 to 100 μm. Further, in a case where rare cells such as circulating tumor cells in blood are to be trapped, the short width is preferably from 4 to 10 μm. Within such a range, cells in the blood pass through the through-holes, and rare cells can effectively be trapped on the substrate. Here, the short width means, in a case where the cross sectional shape is rectangular, its short side, and in a case where it is elliptic, the width between parallel lines with the shortest distance among pairs of parallel lines in contact with the ellipse.

Here, the cross sectional shape, the average diameter and the average short width of the through-holes are measured by observation with an optical microscope, a lase microscope or an electron microscope.

The interval (pitch) between a through-hole and a through-hole adjacent to the through-hole formed on the substrate is, from the viewpoint of the number of holes which can be arranged, the filter strength and the observation of an object after trapped, preferably from 4 to 200 μm, more preferably from 7 to 30 μm.

The aperture of the substrate is, with a view to reducing the pressure difference caused between above and below the filter, preferably from 5 to 70%, more preferably from 15 to 65%. Here, the aperture of the substrate is defined as “(opening area/substrate area)×100” and is measured as follows.

A certain region A photographed using an optical microscope or a laser microscope is taken as the substrate area, and the opening area contained in the region A is calculated by image processing based on the contrast.

### (Method 1 for Producing Substrate Having Through-Holes)

In a case where the substrate is made of glass, the through-holes may be formed, for example, by using a laser. An example is shown below. A glass substrate is prepared, and as a laser, for example, a high repetition rate pulse laser (wavelength: 355 nm, repetition frequency: 110 kHz, 28 W) emitted from a third harmonic Nd: YVO4 laser apparatus is used. The lase pulse (pulse width: 20 ns, power: 7 W, beam diameter: 3.5 mm) emitted from the laser apparatus is focused on the surface of the glass substrate by an object lens. The irradiation time per through-hole is about 3.5 ms, the glass substrate is fixed on an XY stage, and the XY stage is optionally moved every time the through-hole is processed, whereby a group of two-dimensionally arranged 10×10 through-holes with a pitch of 200 μm can be formed.

### (Method 2 for Producing Substrate Having Through-Holes)

In a case where the substrate is made of a resin, a substrate having through-holes may be formed, for example, by means of dry etching as follows. A Ti metal hard mask is formed on a resin film by sputtering. Then, a resist is patterned in a desired hole shape by photolithography. Then, the Ti hard mask is formed into the same shape as the resist pattern by dry etching with a chlorine gas. Using the patterned Ti film as a mask, dry etching with an oxygen gas is carried out to form through-holes on the resin film. Finally, by dry etching with a chlorine gas, the Ti mask is removed to obtain a transparent perforated resin film.

### (Method for Forming Coating Layer on Substrate)

The substrate constituting the cell-trapping filter of the present invention has, on at least its surface, a coating layer formed by applying the fluorinated polymer (A) which is liquid or in a case where the fluorinated polymer (A) is not liquid, a liquid having the fluorinated polymer (A) dissolved or dispersed in a solvent and then removing the solvent.

The layer formed of the fluorinated polymer (A) may be formed on at least a part of the surface of the substrate constituting the cell-trapping filter. It is usually formed on the substrate surface on the side to be in contact with the cell liquid, however, it may further be formed on the inner wall of the through-holes, and may further be provided on the substrate surface on the side from which the cell liquid is discharged.

The thickness of the layer formed on the surface of the substrate is not particularly limited, and is preferably from 50 nm to 50 μm, more preferably from 100 nm to 2 μm.

### EXAMPLES

Now, the present invention will be described in detail with reference to Examples, but the present invention is not limited by the following description. Ex. 1 to 3, 6, 7, 9 to 14, 16 to 21, 23 to 30, and 31 to 42 are Examples of the present invention, and Ex. 4, 5, 8, 15, and 22, are Comparative Examples.

Further, Ex. 1 to 3, 6, 7, 9 to 14, 16 to 21 and 23 to 30 are Examples of the present invention showing properties such as a low adsorption rate to proteins and cells and excellent durability with respect to the substrate having a coating layer of a fluorinated polymer constituting the cell-trapping filter of the present invention.

### [Copolymer Composition]

20 mg of a fluorinated polymer obtained, was dissolved in chloroform, and the copolymer composition was determined by ¹H-NMR.

### [Fluorine Atom Content]

The fluorine atom content was determined by ¹H-NMR, ion chromatography and elemental analysis.

### [Glass Transition Temperature (Tg)]

The glass transition temperature of a fluorinated polymer was measured by raising or lowering the temperature between −30° C. to 200° C. at a rate of 10° C./min. by DSC (manufactured by TA Instruments). The temperature for a change from the rubber state to the glass state in the second cycle of the temperature lowering, was adopted as the glass transition temperature.

### [Molecular Weight]

The number average molecular weight (Mn), mass average molecular weight (Mw) and molecular weight distribution (mass average molecular weight (Mw)/number average molecular weight (Mn)) of a fluorinated polymer, were measured by means of a GPC device (HLC8220, manufactured by Tosoh Corporation) using tetrahydrofuran (THF) as a solvent. [Proportion P]

The proportion P was calculated by the following formula. The proportion (mass %) of units having a biocompatible group to all units in a fluorinated polymer, was measured by ¹H-NMR (JEOL, Ltd., AL300), ion chromatography (Dionex DX500) and elemental analysis (PerkinElmer Co., Ltd., 2400.CHSN).

Proportion P (%)=(proportion (mass %) of units having biocompatible group to all units in fluorinated polymer/fluorine atom content (mass %))×100

### [Evaluation Methods]

### (Water-Insolubility)

10 mg of a fluorinated polymer used in each Ex. and 1 g of water were weighed into a sample tube and stirred for 1 hour at room temperature, whereupon the water-insolubility was visually confirmed. The evaluation was carried out on the basis of the following standards.

◯ (good): The fluorinated polymer remained.

X (bad): The fluorinated polymer was completely dissolved and did not remain.

### (Protein Non-Adsorption)

### <Protein Non-Adsorption Test>

### (1) Preparation of Coloring Solution and Protein Solution

As the coloring solution, one having 50 mL of a peroxidase color solution (3,3′,5,5′-tetramethylbenzidine (TMBZ), manufactured by KPL, Inc.) and 50 mL of TMB Peroxidase Substrate (manufactured by KPL, Inc.) mixed, was used.

As the protein solution, one having protein (POD-goat anti mouse IgG, manufactured by Bio-Rad Laboratories, Inc.) diluted to 16,000-fold with phosphate buffer solution (D-PBS, manufactured by Sigma Co.), was used.

### (2) Protein Adsorption

To 3 wells of a 24-well polystyrene microplate having a coating layer formed on each well surface, 2 mL of the protein solution was dispensed (using 2 mL per well) and left to stand at room temperature for one hour.

As a blank, the protein solution was dispensed to 3 wells of a 96-well microplate in an amount of 2 μL (using 2 μL per well).

### (3) Washing of Wells

Then, the 24-well microplate was washed four times with 4 mL of phosphate buffer solution (D-PBS, manufactured by Sigma Co.) having 0.05 mass % of a surfactant (Tween 20, manufactured by Wako Pure Chemical Industries, Ltd.) incorporated (using 4 mL per well).

### (4) Dispensing of Coloring Solution

Then, to the washed 24-well microplate, 2 mL of the coloring solution was dispensed (using 2 mL per well), and a coloring reaction was carried out for 7 minutes. The coloring reaction was stopped by adding 1 mL of 2N sulfuric acid (using 1 mL per well).

As the blank, to the 96-well microplate, 100 μL of the coloring solution was dispensed (using 100 μL per well), and a coloring reaction was carried out for 7 minutes. The coloring reaction was stopped by adding 50 μL of 2N sulfuric acid (using 50 μL per well).

### (5) Preparation for Measurement of Absorbance

Then, from each well of the 24-well microplate, 150 μL of the liquid was taken and transferred to the 96-well microplate.

### (6) Measurement of Absorbance and Protein Adsorption Rate Q

As to the absorbance, the absorbance at 450 nm was measured by MTP-810Lab (manufactured by Corona Electric Co., Ltd.). Here, the average value of the absorbance (N=3) of the blank was designated as A0. The absorbance of the liquid transferred from the 24-well microplate to the 96-well microplates was designated as A1. The protein adsorption rate Q1 (%) was obtained by the following formula, and the protein adsorption rate Q was set to be the average value.

Q1=100×A1/{A0×(100/dispensed amount of the protein solution in the blank)}=100×A1/{A0×(100/2 μL)}

### (Durability of Coating Layer)

### (1) Durability of Coating Layer Formed on the Well Surface of Microplate

In each Ex. given below, a 24-well microplate having a coating layer formed on the well surface, was immersed in water of 37° C. for one week, and then heated and dried at 60° C. for 2 hours. Thereafter, the above-described protein non-adsorption test was conducted to measure the protein adsorption rate Q, and the durability of the coating layer was evaluated according to the following standards. Here, the rate of increase in protein adsorption rate Q was calculated by the following formula.

Rate of increase in protein adsorption rate Q (%)=100×{(protein adsorption rate (%) after immersion in water of 37° C. for one week initial protein adsorption rate (%))−1)}

◯ (good): As compared to initial, the rate of increase in protein adsorption rate Q after the immersion is less than 5%.

Δ (acceptable): As compared to initial, the rate of increase in protein adsorption rate Q after the immersion is at least 5% and less than 20%.

X (bad): As compared to initial, the rate of increase in protein adsorption rate Q after immersion is at least 20%.

### (2) Durability of Coating Layer Formed on the Surface of Glass Petri Dish

In each Ex. given below, 6 mL of water was put in a glass petri dish having a coating layer formed on the surface and left at rest for 24 hours in an oven at 40° C. Then, after removing the water, the glass petri dish was heated and dried at 100° C. for 1 hour in an oven. Thereafter, the above-described protein non-adsorption test was conducted to measure the protein adsorption rate 0, and the durability of the coating layer was evaluated according to the following standards. Here, the substrate adhesion rate Z was calculated by the following formula. As the value of the substrate adhesion rate is small, the durability of the coating layer is excellent.

Substrate adhesion rate Z=protein adsorption rate (%) after being left at rest at 40° C. for 24 hours with water put therein÷initial protein adsorption rate (%)

The abbreviations of the raw materials used in the preparation of the fluorinated polymers are shown below.

### (Monomers)

C6FMA: CH₂═C(CH₃)COO(CH₂)₂(CF₂)₅CF₃.

C6FA: CH₂═CHCOO(CH₂)₂(CF₂)₅CF₃.

C1FMA: CH₂═C(CH₃)COOCH₂CF₃.

CBA: N-Acryloyloxyethyl-N,N-dimethylammonium-α-N-methylcarboxybetaine.

CBMA: N-Methacryloyloxyethyl-N,N-dimethylammonium-α-N-methyl carboxybetaine.

MPC: 2-Methacryloyloxyethyl phosphorylcholine.

2-EHA: 2-Ethylhexyl acrylate (CH₂═CHCOOCH₂CH(C₂H₅)CH₂CH₂CH₂CH₃).

PEG9A: Polyethylene glycol monoacrylate (EO number average 9) (CH₂═CHCOO(C₂H₄O)₉H).

OMA: Octyl methacrylate (CH₂═C(CH₃)COO(CH₂)₈H).

PEG4.5A: Polyethylene glycol monoacrylate (EO number average 4.5) (CH₂═CHCOO(C₂H₄O)_{4.5}H).

PEPEGA: CH₂═CHCOO(C₂H₄O)₁₀(C₃H₆O)₂₀(C₂H₄O)₁₀H.

MPEG9MA: CH₂═C(CH₃)COO(C₂H₄O)₉CH₃.

PEBMA: CH₂═C(CH₃)COO[(C₂H₄O)₁₀(C₄H₈O)₅]H.

DAEMA: N,N-Dimethylaminoethyl methacrylate.

IMADP: 3,5-Dimethylpyrazole adduct of 2-isocyanatoethyl methacrylate (compound represented by the following formula (7)).

KBM-503: 3-Methacryloyloxypropyl trimethoxysilane (product name “KBM-503”, manufactured by Shin-Etsu Silicone Co., Ltd.).

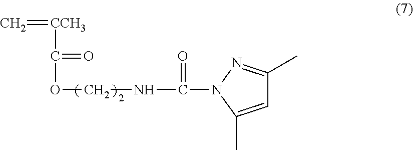

### (Polymerization Initiators)

AIBN: 2,2′-Azobisisobutyronitrile.

VPE: Trade name “VPE-0201” (polymeric azo initiator having the structure (6), manufactured by Wako Pure Chemical Industries, Ltd.).

### (Polymerization Solvent)

EtOH: Ethanol.

MP: 1-Methoxy-2-propanol.

### Production Example 1

0.886 g (3.0 mmol) of MPC and 3.025 g (7.0 mmol) of C6FMA were weighed into a 300 mL three-necked flask, and 0.391 g of AIBN as a polymerization initiator, and 15.6 g of ethanol (EtOH) as a polymerization solvent were added. The molar ratio of C6FMA to MPC was made to be C6FMA/MPC=70/30, the total concentration of the monomers in the reaction solution was made to be 20 mass %, and the initiator concentration was made to be 1 mass %.

Inside of the flask was thoroughly purged with argon, then sealed and heated for 16 hours at 75° C. to conduct a polymerization reaction. The reaction solution was cooled with ice and then, dropped into diethyl ether, to precipitate the polymer. The obtained polymer was sufficiently washed with diethyl ether, and then dried under reduced pressure to obtain a white powdery fluorinated polymer (A-1).

The copolymer composition of the obtained fluorinated polymer (A-1) was measured by ¹H-NMR, and found to be C6FMA units/MPC units=44/56 (molar ratio).

### Production Examples 2 to 15

Polymers in Production Examples 2 to 15 were obtained in the same manner as in Production Example 1 except that the types and charged amounts of monomers, and the type of the polymerization solvent were changed as shown in Table 1.

The charged ratio of monomers, the amount of the polymerization initiator added, the type of the polymerization solvent, and the type, copolymer composition and fluorine atom content in the obtained fluorinated polymer in each of Production Examples 1 to 15, are shown in Table 1.

TABLE 1

| | | | Polymer- | | Fluorinated polymer | | | | |
|---|---|---|---|---|---|---|---|---|---|
| | Charged ratio | | ization | Polymer- | | Copolymer | | Fluorine | |
| Production | of monomers | | initiator | ization | | composition | | atom content | |
| Example | Molar ratio | Mass ratio | [mass %] | solvent | Type | Molar ratio | Mass ratio | [mass %] | Tg [° C.] |
| 1 | C6FMA/MPC | C6FMA/MPC | AlBN 1 | EtOH | A-1 | C6FMA/MPC | C6FMA/MPC | 30.6 | 117 |
| | 70/30 | 77/23 | | | | 44/56 | 54/46 | | |
| 2 | C6FMA/MPC | C6FMA/MPC | AlBN 1 | EtOH | A-2 | C6FMA/MPC | C6FMA/MPC | 28.8 | 147 |
| | 50/50 | 59/41 | | | | 41/59 | 50/50 | | |
| 3 | C6FMA/MPC | C6FMA/MPC | AlBN 1 | EtOH | A-3 | C6FMA/MPC | C6FMA/MPC | 16.0 | 172 |
| | 30/70 | 39/61 | | | | 21/79 | 28/72 | | |
| 4 | C6FMA/MPC | C6FMA/MPC | AlBN 1 | EtOH | X-1 | C6FMA/MPC | C6FMA/MPC | 11.0 | 178 |
| | 20/80 | 27/73 | | | | 14/86 | 19/81 | | |
| 5 | C1FMA | C1FMA | AlBN 1 | MP | X-2 | C1FMA | C1FMA | 33.9 | 196 |
| | 100 | 100 | | | | 100 | 100 | | |
| 6 | C1FMA/MPC | C1FMA/MPC | AlBN 1 | EtOH | A-4 | C1FMA/MPC | C1FMA/MPC | 17.8 | 132 |
| | 70/30 | 57/43 | | | | 66/34 | 53/47 | | |
| 7 | C1FMA/MPC | C1FMA/MPC | AlBN 1 | EtOH | A-5 | C1FMA/MPC | C1FMA/MPC | 13.6 | 147 |
| | 60/40 | 46/54 | | | | 54/46 | 40/60 | | |
| 8 | C1FMA/MPC | C1FMA/MPC | AlBN 1 | EtOH | X-3 | C1FMA/MPC | C1FMA/MPC | 10.5 | 168 |
| | 50/50 | 36/64 | | | | 44/56 | 32/68 | | |
| 9 | C6FMA/CBMA | C6FMA/CBMA | AlBN 1 | EtOH | A-6 | C6FMA/CBMA | C6FMA/CBMA | 39.2 | 121 |
| | 50/50 | 67/33 | | | | 52/48 | 69/31 | | |
| 10 | C6FMA/CBMA | C6FMA/CBMA | AlBN 1 | EtOH | A-7 | C6FMA/CBMA | C6FMA/CBMA | 26.5 | 155 |
| | 30/70 | 46/54 | | | | 30/70 | 46/54 | | |
| 11 | C6FMA/CBMA | C6FMA/CBMA | AlBN 1 | EtOH | A-8 | C6FMA/CBMA | C6FMA/CBMA | 19.1 | 173 |
| | 20/80 | 33/67 | | | | 20/80 | 33/67 | | |
| 12 | C6FMA/CBA | C6FMA/CBA | AlBN 1 | EtOH | A-9 | C6FMA/CBA | C6FMA/CBA | 33.7 | |
| | 50/50 | 59/41 | | | | 50/50 | 59/41 | | |
| 13 | C6FMA/CBA | C6FMA/CBA | AlBN 1 | EtOH | A-10 | C6FMA/CBA | C6FMA/CBA | 27.4 | |
| | 30/70 | 48/52 | | | | 30/70 | 48/52 | | |
| 14 | C6FMA/CBA | C6FMA/CBA | AlBN 1 | EtOH | A-11 | C6FMA/CBA | C6FMA/CBA | 20.0 | |
| | 20/80 | 35/65 | | | | 20/80 | 35/65 | | |
| 15 | CBA | CBA | AlBN 1 | EtOH | X-4 | CBA | CBA | 0 | |
| | 100 | 100 | | | | 100 | 100 | | |

### Production Example 16

5 g (11.6 mmol) of C6FMA was weighed into a 300 mL three-necked flask, and 0.7 g of VPE as a polymerization initiator and 13.3 g of MP as a polymerization solvent were added. The total concentration of monomers in the reaction solution was made to be 30 mass %, and the charged molar ratio of C6FMA to VPE was made to be C6FMA/VPE=97/3.

Inside of the flask was thoroughly purged with argon, and then, sealed and heated for 16 hours at 75° C. to conduct a polymerization reaction. The reaction solution was cooled with ice and then dropped into diethyl ether to precipitate the polymer. The obtained polymer was sufficiently washed with diethyl ether and then dried under reduced pressure to obtain a white powdery fluorinated polymer (A-12).

### Production Examples 17 to 19

Each polymer was obtained in the same manner as in Production Example 16 except that the types of monomers, and the charged ratio of monomers to a polymerization initiator, were changed as shown in Table 2.

The types and charged ratios of monomers and polymerization initiator, the type of the polymerization solvent, as well as the type, copolymer composition and fluorine atom content of the obtained fluorinated polymer in each of Production Examples 16 to 19, are shown in Table 2. Here, “NA” in Table 2 means that the glass transition temperature was not detected.

TABLE 2

| | Charged ratio | | | Fluorinated polymer | | | | |
|---|---|---|---|---|---|---|---|---|
| | (Polymerization | | | | Copolymer | | Fluorine atom | |
| Production | initiator/monomer) | | Polymerization | | composition | | content | |
| Example | Molar ratio | Mass ratio | solvent | Type | Molar ratio | Mass ratio | [mass %] | Tg [° C.] |
| 16 | C6FMA/VPE | C6FMA/VPE | MP | A-12 | C6FMA/VPE | C6FMA/VPE | 50.3 | NA |
| | 97/3 | 88/12 | | | 97/3 | 88/12 | | |
| 17 | C6FMA/VPE | C6FMA/VPE | MP | A-13 | C6FMA/VPE | C6FMA/VPE | 44.6 | NA |
| | 94/6 | 78/22 | | | 94/6 | 78/22 | | |
| 18 | C6FMA/VPE | C6FMA/VPE | MP | A-14 | C6FMA/VPE | C6FMA/VPE | 40 | NA |
| | 92/8 | 70/30 | | | 92/8 | 70/30 | | |
| 19 | C1FMA/VPE | C1FMA/VPE | MP | A-15 | C1FMA/VPE | C1FMA/VPE | 23.8 | NA |
| | 97/3 | 70/30 | | | 97/3 | 70/30 | | |

### Production Example 20

In a 100 mL pressure-resistant glass bottle, 40 g of 2-EHA, 40 g of PEG9A, 0.66 g of V-601 (oil-soluble azo polymerization initiator, manufactured by Wako Pure Chemical Industries, Ltd.) and 49.8 g of m-xylene hexafluoride (manufactured by Central Glass Co., Ltd., hereinafter referred to as “m-XHF”) were charged, and then, sealed and heated for 16 hours at 70° C. To this reaction solution, 20 g of C6FA, 40 g of m-XHF and 0.48 g of V-601 were charged, and then, sealed and heated for 16 hours at 70° C., to obtain a fluorinated polymer (A-16). The copolymer composition of the fluorinated polymer (A-16) was measured. As a result, it was found to be a fluorinated polymer having PEG9A units, C6FA units and 2-EHA units in a molar ratio of 24:14:62 (mass ratio of 40:20:40). As a result of measurement of the molecular weight, the number average molecular weight (Mn) of the fluorinated polymer (A-16) was 17,000, the mass average molecular weight (Mw) was 40,000, and the molecular weight distribution (mass average molecular weight (Mw)/number average molecular weight (Mn)) was 2.3. [Production Example 21]

In a 100 mL pressure-resistant glass bottle, 15 g of OMA, 35 g of PEG4.5A, 0.41 g of V-601 and 31.3 g of m-XHF, were charged, and then, sealed and heated for 16 hours at 70° C. To this reaction solution, 50 g of C6FMA, 100 g of m-XHF and 1.2 g of V-601, were charged, and then sealed and heated for 16 hours at 70° C., to obtain a fluorinated polymer (A-17).

The copolymer composition of the fluorinated polymer (A-17) was measured. As a result, it was confirmed to be a fluorinated polymer having PEG4.5A units, C6FMA units and OMA units in a molar ratio of 40:36:24 (mass ratio of 35:50:15).

### Production Example 22

In a 100 mL pressure-resistant glass bottle, 80 g of PEPEGA, 0.66 g of V-601 and 49.8 g of m-XHF, were charged, and then, sealed and heated for 16 hours at 70° C. To this reaction solution, 20 g of C6FA, 40 g of m-XHF and 0.48 g of V-601, were charged, and then, sealed and heated for 16 hours at 70° C., to obtain a fluorinated polymer (A-18). The copolymer composition of the fluorinated polymer (A-18) was measured. As a result, it was confirmed to be a fluorinated polymer having PEPEGA units and C6FA units in a molar ratio of 44:56 (mass ratio of 80:20).

### Production Example 23

10.8 g (54 parts by mass) of C6FMA, 5.2 g (26 parts by mass) of MPEG9MA, 3.2 g (16 parts by mass) of PEBMA, 0.4 g (2 parts by mass) of DAEMA, 0.4 g (2 parts by mass) of IMADP, 59.8 g of acetone as a polymerization solvent and 0.2 g (1 part by mass) of 4,4′-azobis(4-cyanovaleric acid) as a polymerization initiator, were charged, and while shaking in a nitrogen atmosphere, polymerization was conducted at 65° C. for 20 hours, to obtain a pale yellow solution (polymer solution containing a fluorinated copolymer (A-19)).

The copolymer composition of the fluorinated polymer (A-19) was measured. As a result, it was confirmed to be a fluorinated polymer having C6FMA units, PEBMA units, MPEG9MA units, DAEMA units and IMADP units in a molar ratio of 59:24:8:6:4 (mass ratio of 54:26:16:2:2).

### Ex. 1

The fluorinated polymer (A-1) obtained in Production Example 1 was dissolved in ethanol so that its concentration would be 0.05 mass %, to prepare a coating solution. The coating solution was dispensed in an amount of 2.2 mL on a 24-well microplate (microplate for suspension culture (not surface-treated) 24 wells, manufactured by AGC TECHNO GLASS CO., LTD.) and left to stand for 3 days to evaporate the solvent, thereby to form a coating layer on the well surface.

### Ex. 2 to 19

A coating solution was prepared in the same manner as in Ex. 1 except that a polymer shown in Table 3 was used instead of the fluorinated polymer (A-1). Further, by using the coating solution, in the same manner as in Ex. 1, a coating layer was formed on the well surface of a 24-well microplate.

### Ex. 20 to 23

A coating solution was prepared in the same manner as in Ex. 1 except that a fluorinated polymer shown in Table 3 was used instead of the fluorinated polymer (A-1). Further, by using the coating solution, in the same manner as in Ex. 1, a coating layer was formed on the well surface of a 24-well microplate.

### Ex. 24 to 26

To a solution prepared by dissolving the fluorinated polymer (A-16) obtained in Production Example 20 in AC6000 (manufactured by Asahi Glass Company, Limited) so that its concentration would be 0.05 mass %, a crosslinking agent was added to prepare a coating solution. As the crosslinking agent, to 28 g of the above solution, 0.1 mg of hexamethylene diisocyanate in Ex. 24, 0.13 mg of isophorone diisocyanate in Ex. 25, and 0.1 mg of TLA-100 (manufactured by Asahi Kasei Corporation) in Ex. 26, were added. By using such a coating solution, in the same manner as in Ex. 1, a coating layer was formed on the well surface of a 24-well microplate.

The type, fluorine atom content and proportion P of the fluorinated polymer contained in the coating solution in each Ex. as well as the evaluation results of the water-insolubility and protein non-adherent properties, are shown in Table 3.

TABLE 3

| | | | | | | Durability of coating layer | | |
|---|---|---|---|---|---|---|---|---|
| | Fluorinated polymer | | | | Protein non- | Adsorption | Rate of | |
| | | Fluorine | | | adsorption | rate Q | increase in | |
| | | atom | | | adsorption | (after | adsorption | |
| | | content | Proportion | Water | rate Q | immersion) | rate Q | Evaluation |
| Ex. | Type | [mass %] | P [%] | insolubility | [%] | [%] | [%] | result |
| 1 | A-1 | 30.6 | 1.5 | ◯ | 0.033 | 0.035 | 6.06 | Δ |
| 2 | A-2 | 28.8 | 1.7 | ◯ | 0.079 | | | |
| 3 | A-3 | 16.0 | 4.5 | ◯ | 0 | | | |
| 4 | X-1 | 11.0 | 7.4 | X | — | | | |
| 5 | X-2 | 33.9 | 0 | ◯ | 0.3 | | | |
| 6 | A-4 | 17.8 | 2.6 | ◯ | 0.035 | | | |
| 7 | A-5 | 13.6 | 4.4 | ◯ | 0.082 | | | |
| 8 | X-3 | 10.5 | 6.5 | X | — | | | |
| 9 | A-6 | 39.2 | 0.8 | ◯ | 0.092 | | | |
| 10 | A-7 | 26.5 | 2.0 | ◯ | 0.021 | | | |
| 11 | A-8 | 19.1 | 3.5 | ◯ | 0 | | | |
| 12 | A-9 | 33.7 | 1.2 | ◯ | 0.03 | | | |
| 13 | A-10 | 27.4 | 1.9 | ◯ | 0.024 | | | |
| 14 | A-11 | 20.0 | 3.3 | ◯ | 0 | | | |
| 15 | X-4 | 0 | Infinite | X | — | | | |
| 16 | A-12 | 50.3 | 0.2 | ◯ | 0.037 | | | |
| 17 | A-13 | 44.6 | 0.5 | ◯ | 0 | | | |
| 18 | A-14 | 40.0 | 0.7 | ◯ | 0.014 | | | |
| 19 | A-15 | 23.8 | 1.3 | ◯ | 0 | | | |
| 20 | A-16 | 11.8 | 3.4 | ◯ | 0.099 | 0.117 | 18.18 | Δ |
| 21 | A-17 | 28.6 | 1.2 | ◯ | 0.006 | | | |
| 22 | A-18 | 11.8 | 6.8 | X | — | | | |
| 23 | A-19 | 31.9 | 1.3 | ◯ | 0.012 | 0.014 | 16.67 | Δ |
| 24 | A-16 | 11.8 | 3.4 | ◯ | 0.015 | 0.016 | 6.67 | ◯ |
| 25 | A-16 | 11.8 | 3.4 | ◯ | 0.020 | 0.021 | 5.00 | ◯ |
| 26 | A-16 | 11.8 | 3.4 | ◯ | 0.025 | 0.025 | 0.00 | ◯ |

As shown in Table 3, in Ex. 1 to 3, 6, 7, 9 to 14, 16 to 21 and 23, wherein a coating solution containing a fluorinated polymer (A) which has units having a biocompatible group, and a proportion P of from 0.1 to 4.5%, was used, protein was less likely to be adsorbed on the surface, cells were less likely to adhere to the surface, and the biocompatibility was excellent. Further, the fluorinated polymer was hardly soluble in water and thus was excellent in water insolubility.

On the other hand, in Ex. 4, 8, 15 and 22, wherein a polymer having a proportion P exceeding 4.5% was used, the polymer was likely to be easily dissolved in water and thus was insufficient in water resistance. Further, in Ex. 5 wherein a polymer having a proportion P of less than 0.1% was used, protein was adsorbed on the surface, and further, cells adhered to the surface, and thus the biocompatibility was insufficient.

Further, in Ex. 24 to 26 wherein a coating solution having a fluorinated polymer (A) and a crosslinking agent used in combination was used, as compared to Ex, 1, 20 and 23 wherein a crosslinking agent was not used in combination, even after immersion in water of 37° C. for one week, the rate of increase in protein adsorption rate Q was kept to be small, and thus, the durability was excellent.

### Production Example 24

1.48 g (5.0 mmol) of MPC, 1.73 g (4.0 mmol) of C6FMA and 0.25 g (1.0 mmol) of KBM-503 (trimethoxysilyl propyl methacrylate) were weighed into a 300 mL three-necked flask, and 0.346 g of AIBN as a polymerization initiator, and 13.8 g of ethanol (EtOH) as a polymerization solvent, were added. The molar ratio of MPC, C6FMA and KBM-503 was adjusted to be MPC/C6FMA/KBM-503=50/40/10, the total concentration of the monomers in the reaction solution was made to be 20 mass %, and the initiator concentration was made to be 1 mass %.

Inside of the flask was thoroughly purged with argon, and then sealed and heated for 16 hours at 75° C. to conduct a polymerization reaction. The reaction solution was cooled with ice and then dropped into diethyl ether to precipitate the polymer. The obtained polymer was sufficiently washed with diethyl ether and then dried under reduced pressure to obtain a white powdery fluorinated polymer (A-20).

The copolymer composition of the obtained fluorinated polymer (A-20) was measured by ¹H-NMR and found to be MPC units/C6FMA units/KBM-503 units=50/40/10 (molar ratio).

### Production Examples 25 to 27

Each polymer was obtained in the same manner as in Production Example 24 except that the charged ratio of monomers was changed as shown in Table 4.

TABLE 4

| | | Fluorinated polymer | | | |
|---|---|---|---|---|---|
| | | | | Fluorine | |
| | | | Copolymer | atom | |
| Production | Charged ratio | | composition | content | Tg |
| Example | Molar ratio | Type | Molar ratio | [mass %] | (° C.) |
| 24 | MPC/C6FMA/ | A-20 | MPC/C6FMA/ | 28.3 | |
| | KBM-503 | | KBM-503 | | |
| | 50/40/10 | | 50/40/10 | | |
| 25 | MPC/C6FMA/ | A-21 | MPC/C6FMA/ | 31.2 | |
| | KBM-503 | | KBM-503 | | |
| | 50/45/5 | | 50/45/5 | | |
| 26 | MPC/C6FMA/ | A-22 | MPC/C6FMA/ | 32.3 | |
| | KBM-503 | | KBM-503 | | |
| | 50/47/3 | | 50/47/3 | | |
| 27 | MPC/C6FMA/ | A-23 | MPC/C6FMA/ | 34.0 | 147 |
| | KBM-503 | | KBM-503 | | |
| | 50/50/0 | | 50/50/0 | | |

### Ex. 27

0.5 g of the fluorinated polymer (A-20) was weighed into a 20 mL vial, and 0.078 g of a 0.1 mass % nitric acid aqueous solution and 9.42 g of ethanol (EtOH) as a hydrolysis solvent, were added, to bring the concentration of the fluorinated polymer (A-20) in the reaction solution to be 5 mass %. That is, by taking the molecular weight per one unit of the fluorinated polymer (A-20) to be, from the actually measured molar ratio at the time of the copolymerization, MPC molecular weight×0.5+C6FMA molecular weight×0.4+KBM-503 molecular weight×0.1=345.34, the amount of water to be added to trimethoxysilyl groups was made to be 3 molar equivalents.

The vial was stirred for 20 hours by a mixing rotor at room temperature, and the fluorinated polymer (A-20) was diluted with ethanol (EtOH) so that its concentration would be 0.05 mass %, to obtain a coating solution. 3.3 mL of the coating solution was applied to a glass petri dish having a diameter of 35 mm. After the application, by a hot plate, condensation was conducted at 120° C. for 2 hours to form a coating layer.

### Ex. 28 to 30

A coating layer was formed on the surface of a glass petri dish in the same manner as in Ex. 1 except that a fluorinated polymer shown in Table 5 was used instead of the fluorinated polymer (A-20).

The type, the fluorine atom content and the proportion P of the fluorinated polymer contained in the coating solution of each Ex., as well as the evaluation results, are shown in Table 5.

TABLE 5

| | | | | | | Durability of | |
|---|---|---|---|---|---|---|---|
| | | | | | Protein | coating layer | |
| | Fluorinated polymer | | | | non- | Adsorption | |
| | | Fluorine | | | adsorption | rate Q | |
| | | atom | | | adsorption | (after | Substrate |
| | | content | Proportion | Water | rate Q | being left | adhesion |
| Ex. | Type | [mass %] | P [%] | insolubility | [%] | at rest) [%] | rate Z |
| 27 | A-20 | 28.3 | 1.5 | ◯ | 0.077 | 0.081 | 1.1 |
| 28 | A-21 | 31.2 | 1.3 | ◯ | 0.023 | 0.018 | 0.8 |
| 29 | A-22 | 32.3 | 1.3 | ◯ | 0.010 | 0.016 | 1.5 |
| 30 | A-23 | 34.0 | 1.2 | ◯ | 0.031 | 0.186 | 6.0 |

As shown in Table 5, in Ex. 27 to 30 wherein a coating solution containing a fluorinated polymer (A) which has units having a biocompatible group and a proportion P of from 0.1 to 4.5 mass %, was used, protein was less likely to be adsorbed on the surface, cells were less likely to adhere to the surface, and thus, the biocompatibility was excellent. Further, in Ex. 27 to 29 wherein a coating solution containing a fluorinated polymer (A) which has units (m7) was used, as compared to Ex. 30 wherein a coating solution containing a fluorinated polymer (A) which contains no units (m7) was used, the water-insolubility was further excellent.

### Ex. 31

On a transparent PET film having a thickness of 12 μm as a substrate, a Ti film was formed by sputtering, and then on the Ti film, a resist was patterned into a desired hole shape by photolithography. And, by dry etching with a chlorine gas, a Ti hard mask was processed into the same shape as the resist pattern, and using the patterned Ti film as a mask, through-holes were formed on the resin film by dry etching with an oxygen gas. Finally, the Ti mask was removed by dry etching with a chlorine gas to obtain a PET film having a plurality of through-holes. The cross section of the through-holes on the obtained PET film was circular, the average diameter of the circular cross section was 7 μm, and the aperture of the film was 15%.

The fluorinated polymer (A-5) obtained in Production Example 7 was dissolved in ethanol so that its concentration would be 1.0 mass % to prepare a solution. In this solution, the above-obtained PET film was immersed for dip coating, followed by drying at room temperature for 18 hours to obtain a PET film having a coating layer on its surface. The thickness of the coating layer was 1 μm.

The obtained film was evaluated with respect to the cancer cell-trapping test, the test for collecting a single cancer cell, and water insolubility. The evaluation results are shown in Table 6.

### Ex. 32 to 37

A PET film having a coating layer on its surface was obtained in the same manner as in Ex. 31 except that a PET film having properties as identified in Table 6 was used instead of the PET film having a diameter of through-holes of 7 μm, and an aperture of 15%. The thickness of the coating layer was 1 μm in each Ex. The evaluation results are shown in Table 6.

### Ex. 38

A perforated PET film having a coating layer on its surface was obtained in the same manner as in Ex. 31 except that the polymer (X-4) obtained in Production Example 15 was used instead of the fluorinated polymer (A-5). The thickness of the coating layer was 1 μm. The evaluation results are shown in Table 6.

### Ex. 39

A perforated PET film having a coating layer on its surface was obtained in the same manner as in Ex. 31 except that the fluorinated polymer (X-3) obtained in Production Example 8 was used instead of the fluorinated polymer (A-5) and that a PET film having an aperture of 20% was used instead of the PET film having an aperture of 15%. The thickness of the coating layer was 1 μm. The evaluation results are shown in Table 6.

### Ex. 40

A perforated PET film having a coating layer on its surface was obtained in the same manner as in Ex. 31 except that the fluorinated polymer (X-2) obtained in Production Example 5 was used instead of the fluorinated polymer (A-5) and that a PET film having an aperture of 20% was used instead of the PET film having an aperture of 15%. The thickness of the coating layer was 1 μm. The evaluation results are shown in Table 6.

### Ex. 41

A PET film which had straight through-holes having an average diameter of the cross sectional shape of 7 μm and had an aperture of 20%, and which had no coating layer formed on its surface, was used as a filter. The evaluation results are shown in Table 6.

### Ex. 42

An ETFE film having a coating layer on its surface was obtained in the same manner as in Ex. 31 except that an ETFE film having properties as identified in Table 6 was used instead of the PET film having a diameter of through-holes of 7 μm and an aperture of 15%. The thickness of the coating layer was 1 μm in each Ex. The evaluation results are shown in Table 6.

### (Cancer Cell Culture and Preparation of Cell Suspension)

Using RPMI culture medium (manufactured by Life technologies, 11875-093), H358 cells (ATCC CRL-5807) were incubated, and the cells were dissociated from the culture plate by trypsinization, and a 3×10⁴ cells/mL cell suspension was prepared. Then, cytological staining was carried out by CellTracker Red CMTPX (manufactured by Life technologies) at 37° C. for 30 minutes.

0.1 mL of the cell suspension was added to 3.65 mL of a phosphate buffer solution (manufactured by Life technologies, 20012-027) to prepare 3.75 mL of a cell suspension containing 200 cancer cells.

### (Cancer Cell Trapping Test)

An apparatus comprising a syringe, a filter holder (manufactured by Millipore, SX0001300) and a syringe pump KDS-210 (manufactured by KD Scientific Inc.) was prepared, and each of the PET films prepared in Ex. 31 to 33 was set to the filter holder to carry out cancer cell trapping test. 3.75 mL of the above-prepared cancer cell suspension and 3.75 mL of a blood sample were introduced to the syringe and administered at a pump rate of 3 mL, whereby the cancer cells were trapped in the filter.

Then, the cancer cells on the filter were observed by a fluorescence microscope (Biorevo BZ-9000, manufactured by KEYENCE CORPORATION), and the number of trapped cells was counted to obtain the trapping rate in accordance with the following formula. The results are shown in Table 6.

Trapping rate (%)=(number of cancer cells recovered on filter/number of cancer cells introduced to blood sample)×100

### (Single Cell Collection Test)

The filter after the cancer cell trapping test was taken out and placed on a slide glass, and 0.1 mL of a phosphate buffer solution was dropped on the filter, whereupon it was examined whether the cells present on the filter could be collected individually by using a picopipette (manufactured by NepaGene Co., Ltd.) under observation with a microscope. Evaluation was made based on the standards ◯: cells could be collected, and x: cells could not be collected. The results are shown in Table 6.

TABLE 6

| | | | | Fluorinated polymer | | | Protein non- | | | |
|---|---|---|---|---|---|---|---|---|---|---|
| | | Hole | | | Fluorine atom | | adsorption | | | |
| | | diameter | Aperture | | content | Proportion P | adsorption rate | Water | Trapping | Cell |
| Ex. | Hole shape | (μm) | (%) | Type | (mass %) | (%) | (%) | insolubility | rate | collection |
| 31 | Circular | 7 | 15 | A-5 | 13.6 | 4.4 | 0.082 | ◯ | 99% | ◯ |
| 32 | Circular | 5 | 20 | A-5 | 13.6 | 4.4 | 0.082 | ◯ | 99% | ◯ |
| 33 | Circular | 6 | 20 | A-5 | 13.6 | 4.4 | 0.082 | ◯ | 99% | ◯ |
| 34 | Circular | 7 | 20 | A-5 | 13.6 | 4.4 | 0.082 | ◯ | 99% | ◯ |
| 35 | Circular | 8 | 20 | A-5 | 13.6 | 4.4 | 0.082 | ◯ | 99% | ◯ |
| 36 | Rectangular | 7 × 5 | 40 | A-5 | 13.6 | 4.4 | 0.082 | ◯ | 99% | ◯ |
| 37 | Circular | 7 | 0.25 | A-5 | 13.6 | 4.4 | 0.082 | ◯ | 50% | ◯ |
| 38 | Circular | 7 | 20 | X-4 | 0 | Infinite | — | X | 99% | X |
| 39 | Circular | 7 | 20 | X-3 | 10.5 | 6.5 | — | X | 99% | X |
| 40 | Circular | 7 | 20 | X-2 | 33.9 | 0 | 0.3 | ◯ | 99% | X |
| 41 | Circular | 7 | 20 | Nil | — | — | 0.25 | — | 99% | X |
| 42 | Circular | 7 | 20 | A-5 | 13.6 | 4.4 | 0.082 | ◯ | 99% | ◯ |

In Ex. 31 to 37 and 42 in which the proportion P of the fluorinated polymer was from 0.1 to 5, the protein adsorption rate was low and the adsorption adhesion to cells was low, whereby the cells could be collected after cell trapping. Whereas in Ex. 38 to 41 in which the proportion P was out of the above range, protein was significantly adsorbed, and collection after cell trapping was impossible. Further, in Ex. 38, the fluorine atom content was 0%, whereby water insolubility was not excellent.

### INDUSTRIAL APPLICABILITY

The cell-trapping filter of the present invention is useful for trapping various cells. Studies have been conducted on early diagnosis of cancer, probability of metastasis and index to evaluation of therapeutic effect, by counting the number of CTCs in the blood, and selection of therapy by analyzing proteins and mRNAs on the CTC surface, and the cell-trapping filter of the present invention is useful for trapping CTCs.

Further, according to the cell-trapping filter of the present invention, it is possible to effectively separate substances which pass through the filter and substances which do not, utilizing the through-holes. For example, the cell-trapping filter of the present invention is useful to separate cells in a cell culture fluid and the culture fluid, to separate serum components, red blood cells and white blood cells in blood, and to separate blood cell components and rare cells (such as CTCs and CAMLs) in blood.

This application is a continuation of PCT Application No. PCT/JP2016/078095, filed on Sep. 23, 2016, which is based upon and claims the benefit of priority from Japanese Patent Application No. 2015-252237 filed on Dec. 24, 2015. The contents of those applications are incorporated herein by reference in their entireties.

What is claimed is:

1. A cell-trapping filter comprising a substrate and a cell-separating mechanism by size, wherein the substrate has, at least on its surface, a layer formed of a fluorinated polymer having units having a biocompatible group, having a fluorine atom content of from 5 to 60 mass % and having a proportion P represented by the following formula of from 0.1 to 5%:
proportion P=(proportion (mass %) of units having biocompatible group to all units in fluorinated polymer/fluorine atom content (mass %) of fluorinated polymer)×100

2. The cell-trapping filter according to claim 1, wherein the proportion P is from 0.1 to 4.5%.

3. The cell-trapping filter according to claim 1, wherein the biocompatible group in the fluorinated polymer is at least one member selected from the group consisting of a group represented by the following formula (1), a group represented by the following formula (2) and a group represented by the following formula (3):
CₙH₂ₙOₘ (1) 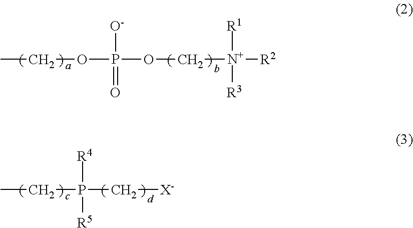
wherein n is an integer of from 1 to 10, m is an integer of from 1 to 100 in a case where the group represented by the formula (1) is contained in a side chain in the fluorinated polymer or from 5 to 300 in a case where contained in the main chain, R¹ to R³ are each independently a C₁₋₅ alkyl group, “a” is an integer of from 1 to 5, b is an integer of from 1 to 5, R⁴ and R⁵ are each independently a C₁₋₅ alkyl group, X⁻ is a group represented by the following formula (3-1) or a group represented by the following formula (3-2), c is an integer of from 1 to 20, and d is an integer of from 1 to 5:

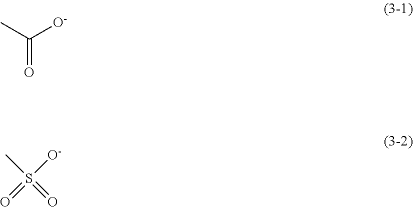

4. The cell-trapping filter according to claim 1, wherein the fluorinated polymer has at least one member selected from the group consisting of units (m1) derived from a monomer represented by the following formula (m1), units (m2) derived from a monomer represented by the following formula (m2) and units (m3) derived from a monomer represented by the following formula (m3): 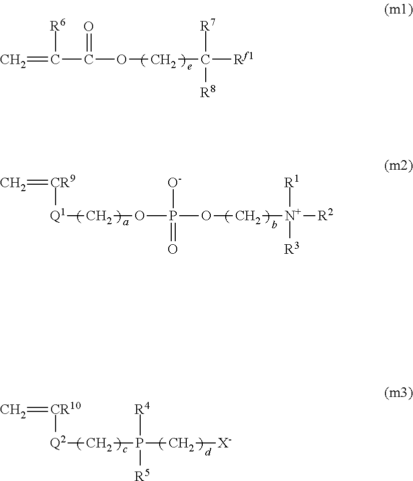
wherein R⁶ is a hydrogen atom, a chlorine atom or a methyl group, e is an integer of from 0 to 3, R⁷ and R⁸ are each independently a hydrogen atom, a fluorine atom or a trifluoromethyl group, R^{f1} is a C₁₋₂₀ perfluoroalkyl group, R⁹ is a hydrogen atom, a chlorine atom or a methyl group, Q¹ is —C(═O)—O— or —C(═O)—NH—, R¹ to R³ are each independently a C₁₋₅ alkyl group, “a” is an integer of from 1 to 5, b is an integer of from 1 to 5, R¹⁰ is a hydrogen atom, a chlorine atom or a methyl group, Q² is —C(═O)—O— or —C(═O)—NH—, R⁴ and R⁵ are each independently a C₁₋₅ alkyl group, X⁻ is a group represented by the following formula (3-1) or a group represented by the following formula (3-2), c is an integer of from 1 to 20, and d is an integer of from 1 to 5;

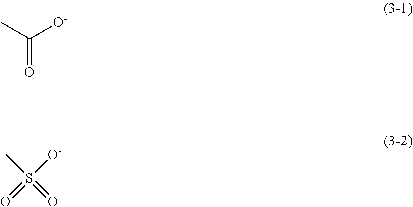

5. The cell-trapping filter according to claim 1, wherein the fluorinated polymer has units (m1) derived from a monomer represented by the following formula (m1) and units (m4) derived from a monomer represented by the following formula (m4): 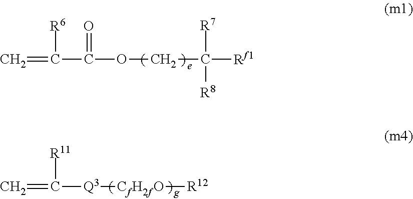
wherein R⁶ is a hydrogen atom, a chlorine atom or a methyl group, e is an integer of from 0 to 3, R⁷ and R⁸ are each independently a hydrogen atom, a chlorine atom or a trifluoromethyl group, R^{f1} is a C₁₋₂₀ perfluoroalkyl group, R¹¹ is a hydrogen atom, a chlorine atom or a methyl group, Q³ is —COO— or —COO(CH₂)ₕ—NHCOO— (wherein h is an integer of from 1 to 4), R¹² is a hydrogen atom or —(CH₂)ᵢ—R¹³ (wherein R¹³ is a C₁₋₈ alkoxy group, a hydrogen atom, a fluorine atom, a trifluoromethyl group or a cyano group, and i is an integer of from 1 to 25), f is an integer of from 1 to 10, and g is an integer of from 1 to 100.

6. The cell-trapping filter according to claim 1, wherein the fluorinated polymer has a segment (I) comprising units (m6) derived from a monomer represented by the following formula (m6) and a segment (II) comprising a molecular chain derived from a polymeric azo initiator having a structure represented by the following formula (6): 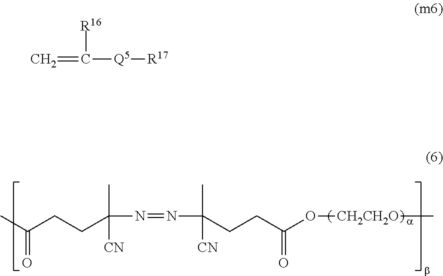
wherein R¹⁶ is a hydrogen atom, a C₁₋₄ alkyl group or a halogen atom, Q⁵ is a single bond or a bivalent organic group, R¹⁷ is a C₁₋₆ polyfluoroalkyl group which may have an etheric oxygen atom between carbon atoms, α is an integer from 5 to 300, and β is an integer of from 1 to 20.

7. The cell-trapping filter according to claim 1, wherein the layer formed of the fluorinated polymer is a layer formed of a fluorinated polymer having hydroxy groups and a crosslinking agent comprising a multifunctional isocyanate compound.

8. The cell-trapping filter according to claim 1, wherein the cell-separating mechanism by size is composed of through-holes.

9. The cell-trapping filter according to claim 8, wherein on the inner wall of at least a part of the through-holes, the layer formed of the fluorinated polymer is formed.

10. The cell-trapping filter according to claim 8, wherein the through-holes have a circular cross section with an average diameter of from 500 nm to 100 μm.

11. The cell-trapping filter according to claim 8, wherein the through-holes have a circular cross section with an average diameter of from 4 to 10 μm.

12. The cell-trapping filter according to claim 8, wherein the through-holes have a rectangular cross section with an average length of the short side of from 4 to 10 μm.

13. The cell-trapping filter according to claim 8, wherein the substrate has a number of through-holes at intervals, and the interval between adjacent through-holes is from 3 to 200 μm.

14. The cell-trapping filter according to claim 1, wherein the substrate has an aperture of from 5 to 70%.

15. The cell-trapping filter according to claim 1, wherein the substrate is made of quartz glass, borosilicate glass, soda lime glass, alkali-free glass, alkali glass or aluminosilicate glass.

16. The cell-trapping filter according to claim 1, wherein the substrate is made of an ethylene/tetrafluoroethylene copolymer (ETFE), a polycarbonate (PC), a polyethylene terephthalate (PET), a polystyrene (PS), a polytetrafluoroethylene (PTFE) or a polyvinylidene fluoride (PVDF).

17. A cell-collecting method, which comprises bringing a suspension containing cells to be trapped into contact with the cell-trapping filter as defined in claim 1 to trap the cells to be trapped on the filter, and recovering the trapped cells.

18. A method for collecting CTCs, which comprises bringing blood into contact with the cell-trapping filter as defined in claim 1 to trap the CTCs on the filter, and recovering the CTCs.
